(19) United States
(12) Patent Application Publication
(10) Pub. No.: US 2015/0240197 A1
(43) Pub. Date: Aug. 27, 2015

(54) CARBONATION SUMP

(71) Applicant: SAPPHIRE ENERGY, INC., San Diego, CA (US)

(72) Inventors: Mehran Parsheh, Las Cruces, NM (US); Jue Zhao, Grand Island, NE (US); Timothy J. Langer, Los Angeles, CA (US)

(73) Assignee: SAPPHIRE ENERGY, INC., SAN DIEGO, CA (US)

(21) Appl. No.: 14/428,295

(22) PCT Filed: Sep. 16, 2013
(86) PCT No.: PCT/US2013/059996

§ 371 Date: Mar. 13, 2015

Related U.S. Application Data
(60) Provisional application No. 61/702,003, filed on Sep. 17, 2012.

(51) Int. Cl. C12M 1/00; C12M 1/06; C12N 1/12; A01G 1/00; A01G 33/00

(52) U.S. Cl. CPC C12M 23/18; A01G 1/001; A01G 33/00; C12N 1/12; C12M 21/02; C12M 29/06; C12M 27/02

(57) ABSTRACT

Disclosed herein is a pond for cultivating a non-vascular photosynthetic organism in a liquid medium, comprising a sump comprising an inflow slope with an angle of about 20 to about 70 degrees, and optionally an outflow slope of with an angle of about 5 to about 40 degrees. The novel sump design results in good overall carbonation efficiency and does not result in a large loss of biomass of a cultivated organism.

### BACKGROUND

Non-vascular photosynthetic organisms, for example, algae and cyanobacteria are capable of rapid growth under a wide range of conditions. As photosynthetic organisms, they have the capacity to transform sunlight into energy that can be used to synthesize a variety of biomolecules for use as industrial enzymes, therapeutic compounds and proteins, nutritional, commercial, or fuel products, etc.

Non-vascular photosynthetic organisms, such as algae and cyanobacteria, can be adapted to in growth in an aqueous environment, and are easily grown in liquid media using light as an energy source. The ability to grow these organisms on a large scale in an outdoor setting, in ponds or other open or closed containers, using sunlight for photosynthesis, enhances their utility for bioproduction, environmental remediation, and carbon fixation.

In order to maximize growth, supplemental carbon dioxide (CO₂) is needed. When growing algae, for example, in an outdoor cultivation pond, CO₂ is added to the liquid media that the algae are growing in. Due to the shallow depth of the pond (for example, six to twelve inches) and the buoyancy of CO₂, a large percentage of CO₂ (for example, up to 75%) that is added to the liquid media may be lost to the atmosphere. Since supplemental CO₂ is one of the biggest production costs when growing algae for industrial purposes, this financial loss is significant.

To circumvent the loss of CO₂, a sump can be placed in the pond which results in a deeper pond depth (for example, 21 to 24 inches) and a higher pressure (psi) resulting in increased uptake of injected CO₂ into the liquid media and so into the organism's biomass.

The depth of the sump cannot be too deep or too much sedimentation of the organism will occur, resulting in an undesirable loss of biomass of the organism and a loss of carbonation efficiency. In addition, mild turbulence is needed in the sump to prevent settling of the microorganisms to the bottom. However, if the turbulence in the sump is too great, a back flow region will appear resulting in a loss of recoverable biomass.

Therefore, there is a need to carefully balance the need for good overall carbonation efficiency with the desire to maintain a degree of turbulence that prevents settling of the microorganism to the bottom of the sump in order to minimize the loss of biomass that can be recovered. The present disclosure meets that need through the novel design of a sump located within a cultivation pond.

### SUMMARY OF THE DISCLOSURE

Provided herein is a pond for the cultivation of a non-vascular photosynthetic organism in a liquid medium, comprising: a) a sump comprising an inflow slope with an angle of from about 20 degrees to about 70 degrees. In some embodiments, the sump further comprises: b) an outflow slope with an angle of from about 5 degrees to about 40 degrees; and c) a back flow region of the inflow slope having a volume that is less than about 50% of the total volume of the sump. In yet other embodiments, the pond and/or sump further comprise a carbon dioxide input that injects CO₂; and the pond has an overall carbonation efficiency of greater than about 20%. In other embodiments, the back flow region of the inflow slope has a volume of less than about 45%, less than about 40%, less than about 35%, less than about 30%, less than about 25%, less than about 20%, less than about 15%, less than about 10%, or less than about 5% of the total volume of the sump. In another embodiment, the back flow region of the inflow slope has a volume that is less than about 25% of the total volume of the sump. In one embodiment, the inflow slope has an angle of about 30 degrees and the outflow slope has an angle of about 20 degrees. In other embodiments, the inflow slope has an angle of from about 20 degrees to about 60 degrees, from about 20 degrees to about 40 degrees, about 20 degrees, about 25 degrees, about 30 degrees, about 35 degrees, about 40 degrees, about 45 degrees, about 50 degrees, about 55 degrees, about 60 degrees, about 65 degrees, or about 70 degrees. In yet other embodiments, the outflow slope has an angle of from about 20 degrees to about 40 degrees. In some embodiments, the pond has an overall carbonation efficiency of greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, or greater than about 95%. In other embodiments, the pond has an overall carbonation efficiency of from about 73% to about 94%, from about 40% to about 90%, from about 81% to about 88%, from about 73% to about 93%, from about 30% to about 70%, about 60%, or about 89%. In one embodiment, the pond has an overall carbonation efficiency of greater than about 31%. In other embodiments, the sump has a depreciation depth of from about 10 inches to about 30 inches. In other embodiments, the sump has a depreciation depth of about 21 inches or about 25 inches. In other embodiments, the sump has a water depth of from about 8 inches to about 40 inches. In other embodiments, the sump has a water depth of about 21 inches, about 30 inches, or about 31 inches. In another embodiment, the pond further comprises a fluid distribution system located upstream of the inflow slope. In one embodiment, the fluid distribution system is a paddle wheel. In some embodiments, the paddle wheel is from about 2 feet to about 200 feet upstream of the inflow slope. In other embodiments, the paddle wheel is about 2 feet, about 4 feet, about 6 feet, about 8 feet, about10 feet, about 12 feet, about 14 feet, about 16 feet, about 18 feet, about 20 feet, about 22 feet, about 24 feet, about 26 feet, about 28 feet, or about 30 feet upstream of the inflow slope. In one embodiment, the fluid distribution system is a jet circulation system. In one embodiment, the liquid medium is a media. In one embodiment, the media further comprises a non-vascular photosynthetic organism. In yet another embodiment, the non-vascular photosynthetic organism is an alga. In other embodiments, the alga is a Chlamydomonas, Volvacales, Nannochloropsis, Desmodesmus, Scenedesmus, Chlorella, Volvox, Arthrospira, Spirulina, Botryococcus, Desmid, Vannochloropsis, or Hematococcus species. In one embodiment, the non-vascular photosynthetic organism is a cyanobacterium. In yet other embodiments, the cyanobacterium is a Synechococcus, Spirulina, Synechoeystis, Athrospira, Prochlorococcus, Chroococcus, Gleoecapsa, Aphanocapsa, Aphanothece, Merismopedia, Microcystis, Coelosphaerium, Prochlorothrix, Oscillatoria, Trichodesmium, Microcoleus, Chroococcidiopisis, Anabaena, Aphanizomenon, Cylindrospermopsis, Cylindrospermum, Tolypothrix, Leptolyngbya, Lyngbya, or Scytonema species. In some embodiments, the sump has a first length (L) of from about 2 feet to about 1000 feet. In other embodiments, the sump has a second length (L₁) of from about 2 feet to about 1000 feet. In yet other embodiments, L is about 10 feet to about 100 feet, and/or L₁ is about 10 feet to about 100 feet. In yet other embodiments, L is about 10 feet, about 12 feet, about 15 feet, about 20 feet, about 25 feet, about 30 feet, about 50 feet, about 100 feet, about 200 feet, or about 500 feet. In other embodiments, L₁ is about 18 feet, about 20 feet, about 23 feet, about 28 feet, about 33 feet, about 38 feet, about 58 feet, about 108 feet, about 208 feet, or about 508 feet. In some embodiments, the sump has a width of about 4 feet, about 6 feet, about 8 feet, about 10 feet, about 12 feet, about 14 feet, about 16 feet, about 18 feet, about 20 feet, about 22 feet, about 24 feet, about 26 feet, about 28 feet, about 30 feet, about 32 feet, about 34 feet, about 36 feet, about 38 feet, or about 40 feet. In other embodiments, the sump has a width of about 8 feet 8 inches, about 15 feet, or about 26 feet 2 inches. In one embodiment, the pond has at least two channels. In other embodiments, each of the pond channels has a length of from about 20 feet to about 2 miles, or from about 50 feet to about 1.5 miles. In other embodiments, each of the pond channels has a length of about 218 feet, about 623 feet, about 636 feet, about 1279 feet or about 1288 feet. In one embodiment, the width of each of the pond channels is measured mid way up or near the top of a side wall of the channel. In another embodiment, the width of each of the pond channels is measured at the bottom of a side wall of the channel. In some embodiments, each of the pond channels has a width of from about 1 foot to 100 feet, or from about 2 feet to about 20 feet. In other embodiments, each of the pond channels has a width of from about 16 feet to about 39 feet. In some embodiments, the pond has an area of from about 25 square feet to about 500 acres. In other embodiments, the pond has an area of from about 50 square feet to about 100 acres. In yet other embodiments, the pond has an area of about 6,400 square feet or about 600 square meters. In some embodiments, the pond has an area of from about 36 square feet to about 500 acres. In other embodiments, the pond has an area of about 0.33 acres, about 1.1 acre, or about 2.2 acres. In other embodiments, the pond is a raceway pond, a single channel pond, a flume pond, a serpentine pond, or a circular pond. In some embodiments, the liquid medium in the pond has a pH of from about 8.0 to about 11.0, a pH of from about 8.8 to about 9.2, or a pH of about 9 or about 10. In one embodiment, the sump has two or more inflow slopes each with an angle of from about 20 degrees to about 70 degrees. In some embodiments, the pond comprises a carbon dioxide input resulting in a carbon dioxide flow rate of from about 0.0022 lb/hr/feet to about 1.15 lb/hr/feet, from about 0.0022 to about 2.0 lb/hr/feet, from about 0.21 lb/hr/feet, to about 1.15 lb/hr/feet, from about 0.26 lb/hr/feet to about 1.0 lb/hr/feet, of about 0.87 lb/hr/feet, of about 0.21 lb/hr/feet, or of from about 0.001 lb/hr/feet to about 2.0 lb/hr/feet. In one embodiment, the carbon dioxide input is a diffuser. In another embodiment, the diffuser is at least one diffuser hose. In another embodiment, the at least one diffuser hose is located along the length (L) of the sump. In another embodiment, the at least one diffuser hose is located along the width of the sump. In yet another embodiment, the at least two diffuser hoses are parallel to each other. In one embodiment, the carbon dioxide input is a nozzle. In one embodiment, the overall carbonation efficiency of the sump is about 87%, the carbon dioxide input results in a carbon dioxide flow rate of about 1.15 lb/hr/feet, the water depth of the sump is about 30 inches, and the liquid has a pH of about 10. In another embodiment, the overall carbonation efficiency of the sump is about 60%, the carbon dioxide input results in a carbon dioxide flow rate of about 0.87 lb/hr/feet, the water depth of the sump is about 30 inches, and the liquid has a pH of about 8.8 to about 9.2. In some embodiments, the pond comprises at least two sumps, wherein each sump has an inflow slope with an angle of from about 20 degrees to about 70 degrees and an outflow slope with an angle of from about 5 degrees to about 40 degrees.

Also described herein is a method of growing a non-vascular photosynthetic organism, comprising: a) obtaining the non-vascular photosynthetic organism; b) obtaining a cultivation pond as described in paragraph 8 above and/or as described throughout the specification; and c) growing the non-vascular photosynthetic organism in the pond. In one embodiment, the non-vascular photosynthetic organism is an alga. In some embodiments, the alga is a Chlamydomonas, Volvacales, Dunaliella, Nannochloropsis, Desmodesmus, Scenedesmus, Chlorella, Volvox, Arthrospira, Spirulina, Botryococcus, Desmid, Vannochloropsis, or Hematococcus species. In another embodiment, the non-vascular photosynthetic organism is a cyanobacterium. In other embodiments, the cyanobacterium is a Synechococcus, Spirulina, Synechocystis, Athrospira, Prochlorococcus, Chroococcus, Gleoecapsa, Aphanocapsa, Aphanothece, Merismopedia, Microcystis, Coelosphaerium, Prochlorothrix, Oscillatoria, Trichodesmiumi, Microcoleus, Chroococcidiopisis, Anabaena, Aphanizomenon, Cylindrospermopsis, Cylindrospermum, Tolypothrix, Leptolyngbya, Lyngbya, or Scytonema species.

Provided herein is a method of measuring percent overall carbonation efficiency of a liquid in a cultivation pond, comprising calculating a formula of:

$$\frac{( {DIC}_{f} - {DIC}_{0} ) * V}{{C}_{T}} \times 100$$

wherein DIC_{f} is dissolved inorganic carbon concentration in the cultivation pond at time finish; DIC₀ is dissolved inorganic carbon concentration in the cultivation pond at time 0 before injection with CO₂; V is volume of liquid in the cultivation pond; and C_{T} is total carbon injected into the cultivation pond.

Also provided herein is a method of measuring percent overall carbonation efficiency of biomass in a cultivation pond, comprising calculating a formula of:

$$\frac{\begin{matrix}{TOC}_{T} \times ( ( {H}_{T} \times 25.4 - 30 ) \div 0.0017 + 12537.8 ) - \\ {TOC}_{0} \times ( ( {H}_{0} \times 25.4 - 30 ) \div 0.0017 + 12537.8 )\end{matrix}}{{}_{0}^{T}{ΣV}{}_{iCO 2}^{} \times 1.835 \times 1000 \div 44 \div 12} \times 100$$

wherein is experiment duration; Vᵢ is CO₂ total injection volume at day of injection, H₀ is cultivation pond water depth at start day (day 0); H_{T} is cultivation pond water depth at finish day (day T); TOC₀ is total organic carbon concentration at day 0; and TOC_{T} is total organic carbon concentration at finish day (day T). In one embodiment, the biomass is a non-vascular photosynthetic organism. In another embodiment, the non-vascular photosynthetic organism is an alga. In other embodiments, the alga is a Chlamydomonas, Volvacales, Dunaliella, Nannochloropsis, Desmodesmus, Scenedesmus, Chlorella, Volvox, Arthrospira, Spirulina, Botryococcus, Desmid, Vannochloropsis, or Hematococcus species. In yet another embodiment, the non-vascular photosynthetic organism is a cyanobacterium. In some embodiments, the cyanobacterium is a Synechococcus, Spirulina, Synechocystis, Athrospira, Prochlorococcus, Chroococcus, Gleoecapsa, Aphanocapsa, Aphanothece, Merismopedia, Microcystis, Coelosphaerium, Prochlorothrix, Oscillatoria, Trichodesmium, Microcoleus, Chroococcidiopisis, Anabaena, Aphanizomenon, Cylindrospermopsis, Cylindrospermum, Tolypothrix, Leptolyngbya, Lynghya, or Scytonema species.

Also provided herein is a pond for the cultivation of a non-vascular photosynthetic organism in a liquid medium, comprising: a) a sump comprising an inflow slope with an angle of from about 20 degrees to about 70 degrees; and b) a back flow region of the inflow slope wherein percent of total surface area of the back flow region is less than percent of total surface area of a back flow region resulting from an inflow slope with an angle of 90 degrees. In some embodiments, the sump further comprises an outflow slope with an angle of from about 5 degrees to about 40 degrees. In other embodiments, the inflow slope has an angle of about 30 degrees, about 45 degrees, or about 60 degrees. In another embodiment, the sump further comprises, a carbon dioxide input that injects CO₂, and the cultivation pond has an overall carbonation efficiency of greater than about 20%.

### BRIEF DESCRIPTION OF THE DRAWINGS

These and other features, aspects, and advantages of the present disclosure will become better understood with regard to the following description, appended claims and accompanying figures where:

FIG. 1 shows overall CO₂ efficiency in a cultivation pond without a sump. The y-axis represents accumulative CO₂ transfer efficiency and the x-axis is CO₂ transfer efficiency (CO₂ carbon to biomass carbon).

FIG. 2 shows a sump located within a cultivation pond.

FIG. 3 shows a portion of a sump located within a cultivation pond and describes the fluid flow over an exemplary inflow slope.

FIG. 4 shows an exemplary raceway pond design with a sump, paddle wheel, and diffuser.

FIG. 5 shows one exemplary configuration of diffusers in a raceway pond.

FIG. 6 shows another exemplary configuration of diffusers in a raceway pond.

FIG. 7 shows yet another exemplary configuration of diffusers in a raceway pond. In addition, two pressure gauges are indicated by solid circles.

FIG. 8 shows the overall carbonation efficiency of the P. P. AQUATECH and SOLVOX®-B diffusers (measured versus calculated). The y-axis is % overall carbonation efficiency and the x-axis is CO₂ flow rate (lb/hr/ft). Aquatech % efficiency measured is represented by a solid diamond, Solvox-B % efficiency measured is represented by a solid square. Aquatech % efficiency calculated is represented by an unfilled diamond, and Solvax-B % efficiency calculated is represented by an unfilled square.

FIG. 9 shows the accumulative overall carbonation efficiency of Aquatech diffusers in a pond that is growing a Desmid (Desmodesmus) species. The CO₂ flow rate flow rate is 0.87 lb/hr/ft. The y-axis is overall carbonation efficiency and the x-axis is the number of days the algal culture was grown.

FIG. 10 is an exemplary raceway cultivation pond and sump.

FIG. 11 is an exemplary raceway cultivation pond and sump.

FIG. 12 is an exemplary raceway cultivation pond and sump described in Example 5.

FIG. 13 is day 5 of a 26 day experiment described in Example 5, showing an average of 0.0 inch of sludge on the bottom of the sump.

FIG. 14 is day 25 of a 26 day experiment described in Example 5, showing an average of 2.033333 inches of sludge on the bottom of the sump.

FIG. 15 is day 26 of a 26 day experiment described in Example 5, showing an average of 2.66667 inches of sludge on the bottom of the sump.

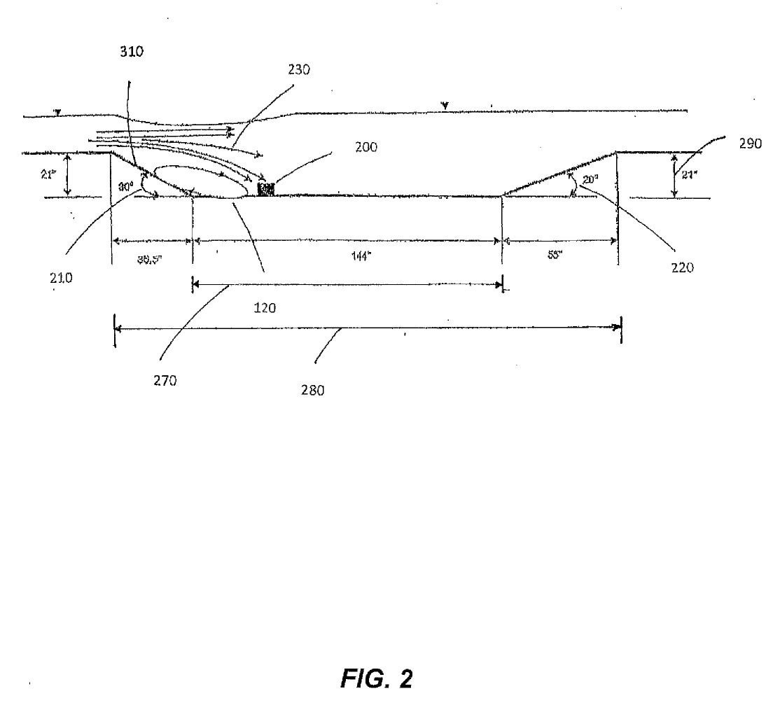

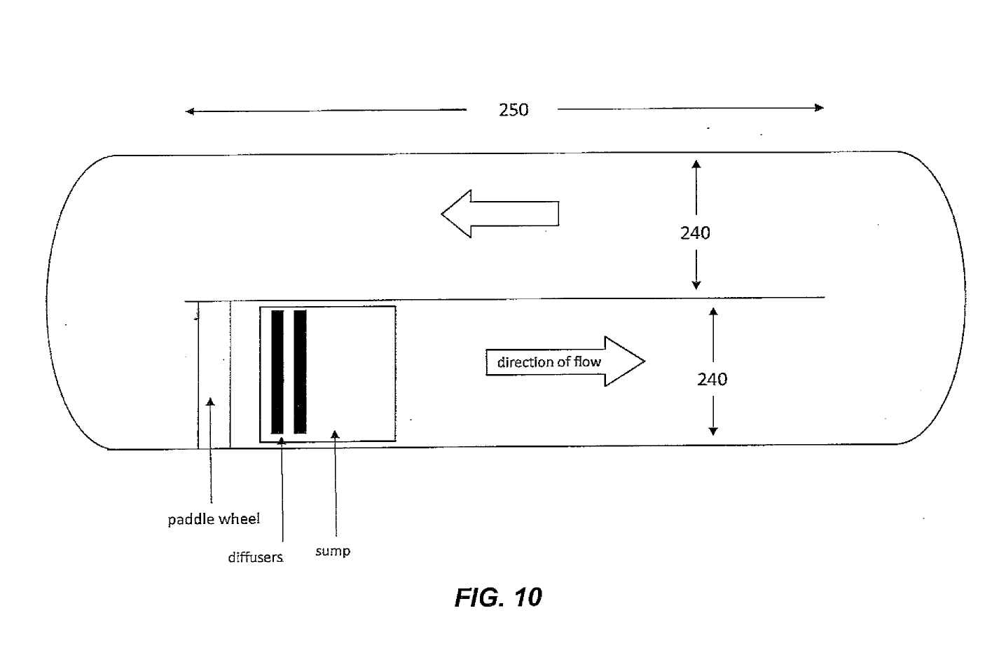

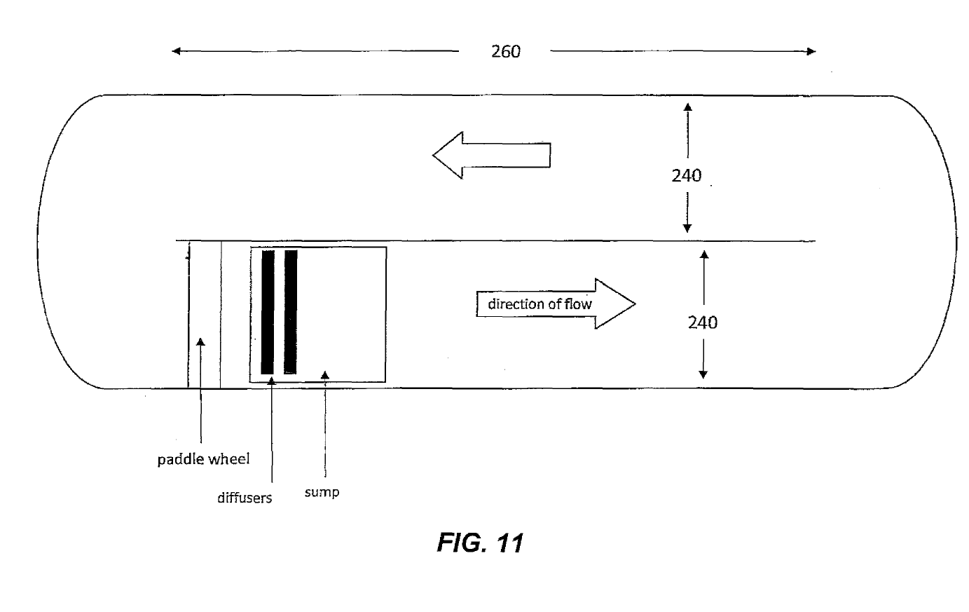

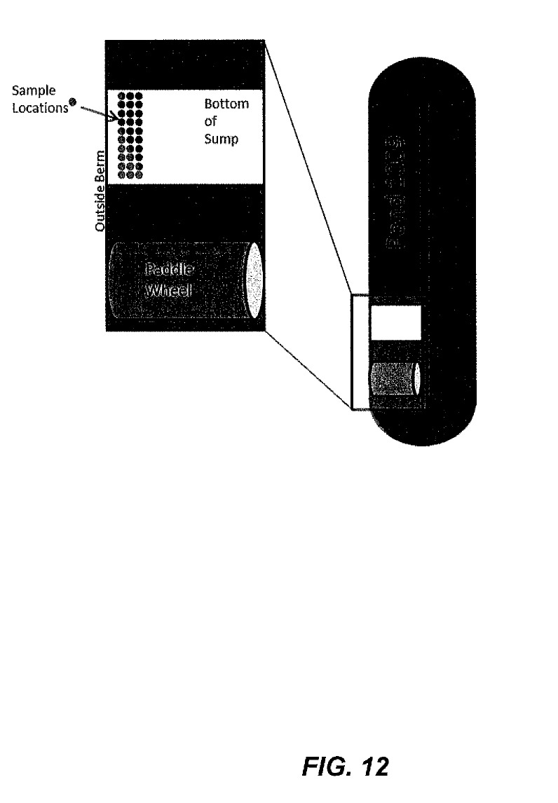

It should be noted that the objects in FIG. 2 to FIG. 7, FIG. 10, FIG. 11, and FIG. 12, are not drawn to scale.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present disclosure.

As used in this specification and the appended claims, the singular forms “a”, “an” and “the” include plural reference unless the context clearly dictates otherwise.

In this disclosure, exemplary ranges of, for example, depths, width, length, area, and volume are provided. It should be understood that the ranges are intended to include sub-ranges, and each incremental amount of depth, width, length, area, and volume in each broad range provided.

Disclosed herein is a novel sump design located within a cultivation pond. One microorganism that can be cultivated or grown in such a pond is an alga, another exemplary microorganism is a cyanobacterium.

Photosynthesis is a chemical process that converts carbon dioxide into organic compounds using the energy from sunlight. The ability to grow or cultivate organisms, such as algae, at commercial scale using sunlight for photosynthesis requires supplemental CO₂ to be added and dissolved into the liquid culture. The algae cultivation pond exploits the natural process of photosynthesis in order to produce algae for high-volume applications, such as the production of biofuels.

CO₂

CO₂ can be injected into the liquid, for example, by diffusers or nozzles placed along, for example, the bottom of the sump. CO₂ can be injected into a return line that goes back into the cultivation pond.

With a diffuser, the CO₂ is injected in gas form into the liquid contained in the sump.

Non-limiting examples of diffusers are: P.P. Aquatech disc diffusers, coarse bubble diffusers, or fine bubble membrane diffusers; Linde Solvox-B perforated diffuser hoses; Porex diffusers; and Aquatech O2b2 diffuser tubing. A diffuser can be a soaker tubing.

A diffuser can be coupled to the bottom of the sump, for example, downstream of the inflow slope (as shown for example by a black box 200 in FIG. 2). A diffuser can be placed/mounted on the bottom of the sump in various configurations known to one skilled in the art.

With a nozzle, the CO₂ is first mixed with a side water flow (flowing at a lower fluid flow rate than the pond fluid flow rate) and the mixture of CO₂ and the side water flow is injected into the liquid contained in the sump through the nozzle.

A nozzle can be coupled to a pressurized fluid source and a carbonation source and configured to generate a jet of carbonated fluid from the pressurized fluid source and the carbonation source. The term “nozzle” and “injector” can be used interchangeably throughout the disclosure. A non-limiting example of a nozzle is a Venturi Nozzle. A Venturi nozzle mixes CO₂ with a side flow and the mixture is then injected into the pond through a nozzle.

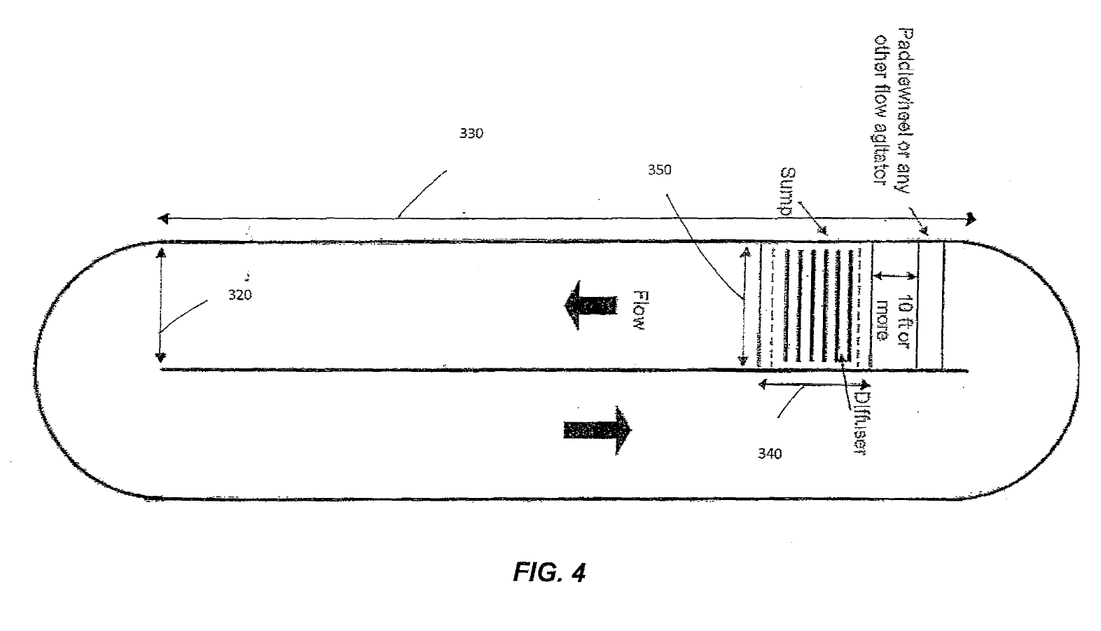

One or more CO₂ input(s) (for example, a nozzle and/or a diffuser) can be placed in a variety of different locations. The CO₂ input(s) can be located in any location and/or configuration in either the pond and/or the sump. Exemplary locations and configurations of diffuser hosing are shown in FIG. 4 to FIG. 7. Carbonation inputs may be coupled to one or more carbonation sources. One row or multiple rows of diffusers can be used in the cultivation pond and/or sump.

In some embodiments, carbonation inputs may substantially span the width of the sump bed. Carbonation inputs may be coupled to the sump bed in a fixed array. Carbonation inputs may be placed in a variety of configurations (e.g., arrays) downstream of the inflow slope. The position of the carbonation inputs relative to the location of the inflow slope may be determined based on various parameters of the inflow slope, such as the height (y-axis) of the angle of the inflow slope, the flow characteristics (e.g., flow velocity), and/or the type of carbonation input.

In some embodiments, the carbonation inputs may be coupled or built into a portion of the inflow slope. The carbonation inputs coupled to the slope may issue jets of carbonated fluid, which in some instances may entrain a co-flow of fluid into the cultivation pond. The jet entrainment may promote the diffusion and/or advection of carbon dioxide into the cultivation pond fluid, yielding a substantially homogeneous mixture downstream from the jets. The resultant flow associated with one or more jets, i.e. jet flow, may induce bulk movement of fluid in the cultivation pond, i.e. circulation, or pond flow.

The optimum carbon dioxide flow rate may be dependent on, for example, the dimensions of the pond, the shape of the pond, the length and width of the sump relative to the pond dimensions, whether there are multiple sumps, the number of diffusers placed in the sump(s), and the type of organism (strain) that is being grown in the cultivation pond. For example, for large ponds with a long distance between two or more of the sumps, the flow rate of CO₂ that is injected into the liquid can be much higher than the case where the distance between the two sumps is shorter. If the sump does not cover the entire pond width, the width of the sump would also be considered when calculating the optimum carbon dioxide flow rate. The optimum carbon dioxide flow rate can be determined by one skilled in the art through field testing of various parameters, for example, as discussed below.

For example, one skilled in the art could set up a “raceway” cultivation pond with one sump (as shown in FIG. 4). One skilled the art might design the cultivation pond such that the length of the sump 340 is 15 feet, the width of the sump 350 is 26 feet 2 inches, the length of each of the channels 330 is 636 feet, and the width of each of the channels at the bottom of the pond is 320 is 32 feet. One skilled in the art could then choose an organism, a solution (media) to culture the organism in, and a range of carbon dioxide flow rates to test, for example, from about 0.21 lb/hr/ft to about 1.15 lb/hr/ft. One skilled in the art could then monitor, for example, the total organic carbon (TOC), total carbon (TC), total dissolved solids (TDS), alkalinity, and pH of the culture, and the health (appearance) and growth rate of the organism and determine if the carbon dioxide flow rate should be decreased or increased to suit the needs of the organism.

The carbon dioxide flow rate can also depend on the length of the diffuser hose. One skilled in the art would know how to calculate the proper flow rate based on the length of the diffuser hose. For example, during a field test, a CO₂ mass flow meter can be used to measure the flow of CO₂ into a diffuser hose. This measurement is in pounds per hour (lb/hr). Then, based on the diffuser length, the CO₂ flow is divided by the length of the diffuser hose, resulting in a CO₂ flow in units of pounds/hour/ft (lb/hr/ft). For example, the CO₂ flow rate can be 26 lb/hr, the length of the diffuser hose is 26 feet, so the flow rate is 1 lb/hr/ft.

The carbon dioxide input can result in, for example, a carbon dioxide flow rate of about 0.21 lb/hr/ft, or about 0.87 lb/hr/ft. Exemplary ranges of carbon dioxide flow rates are from about 0.21 lb/hr/ft to about 1.15 lb/hr/ft, from about 0.26 lb/hr/ft to about 1.0 lb/hr/ft, from about 0.0022 lb/hr/ft to about 2.0 lb/hr/ft, or from about 0.0022 lb/hr/ft to about 3.0 lb/hr/ft. Additional exemplary ranges are: from about 0.0022 lb/hr/ft to about 1.15 lb/hr/ft; or from about 0.001 lb/hr/ft to about 2.0 lb/hr/ft.

The carbonation input is connected to a CO₂ source. The CO₂ source may be, for example, geological CO2, a power plant, a steel mill, a concrete mill, a byproduct of a chemical reaction, or any combination of these. The carbonation source may provide pure carbon dioxide in gaseous form or a mixture of gases including carbon dioxide. Upon introduction of the carbon dioxide to the cultivation pond fluid, at least a portion of the carbon dioxide will dissolve into solution. One skilled in the art will recognize that there are many sources of CO₂ and paths to bring the CO₂ to the cultivation pond.

Measuring Carbonation Efficiency

There are many different ways to measure carbonation efficiency. One skilled in the art could determine the best way to measure carbonation efficiency based on experimental set up. Several examples of methods to calculate carbonation efficiency are provided below.

A method of measuring percent overall carbonation efficiency of a liquid in a cultivation pond, is as follows:

$$\frac{( {DIC}_{f} - {DIC}_{0} ) * V}{{C}_{T}} \times 100$$

wherein DIC_{f} is dissolved inorganic carbon concentration in the cultivation pond at time finish; DIC₀ is dissolved inorganic carbon concentration in the cultivation pond at time 0 before injection with CO₂; V is volume of liquid in the cultivation pond; and C_{T} is total carbon injected into the cultivation pond.

An overall carbonation efficiency calculation is as follows:

$${CO}_{2} transfer efficiency to biomass ( \% ) = \frac{\begin{matrix}{TOC}_{T} \times ( ( {H}_{T} \times 25.4 - 30 ) \div 0.0017 + 12537.8 ) - \\ {TOC}_{0} \times ( ( {H}_{0} \times 25.4 - 30 ) \div 0.0017 + 12537.8 )\end{matrix}}{{}_{0}^{T}{ΣV}{}_{iCO 2}^{} \times 1.835 \times 1000 \div 44 \div 12} \times 100$$

T: experiment duration (days)

Vᵢ: CO₂ total injection volume at day of injection (Standard liter per minute)

H₀: raceway water depth (inches) at start day (day 0)

H_{T}: raceway water depth (inches) at finish day (day T)

TOC₀: total organic carbon concentration (mg/L) at day 0

TOC_{T}: total organic carbon concentration (mg/L) at finish day (day T)

The overall carbonation efficiency of a liquid (e.g., water) with algae can be determined by the following equation:

$$Overall {CO}_{2} efficiency = \frac{( {TC}_{f} - {TC}_{0} ) * V}{{C}_{T}} \times 100$$

Where TC_{f}, TC,₀, and C_{T} are defined as total carbon concentration in the pond at time finish, total carbon concentration at time 0 before CO₂ injection, V is the volume of pond water, and C_{T} is the total carbon injected into the pond.

To determine calculated CO₂ efficiency, two separate methods can be utilized. The first step of the calculation method (method 1) uses the following equation (Mojica, F. J. and Millero, F. J. (2002) Geochimica et Cosmochirnica Acta, Vol. 66, No. 14, pp. 2529-2540) to determine the reaction rate constants (K) for IC formation:

pK₁=−43.6977−0.0129037 S +1.364×10⁻⁴ S²+2885.378/T+7.045159 ln T and (σ=0.010)

pK₂=−452.0940+13.142162 S−8.101×10⁻⁴ S²+21263.61/T+68.483143 ln T +(−581.4428 S +0.259601 S²)/T−1.967035 S ln T

where S=salinity (g/L) and T=temperature (Kelvin). Using the K values and pH, the total inorganic carbon concentration (TCO₂) in solution can be calculated (Mebrach. C., et al. (1973) Limnology and Oceanography, Vol. 18(6)897-907, and Wolf-Gadrow, D. A., et. al. (2007) Marine Chemistry, Vol. 106, pp. 287-300) using the following equation:

$${T CO}_{2} = ( TA ) \frac{{[ {H}^{+} ]}^{2} + {K}_{1} [ {H}^{+} ] + {K}_{1} {K}_{2} [ {H}^{+} ]}{{K}_{1} [ {H}^{+} ] + 2 {K}_{1} {K}_{2}}$$

where TA=total alkalinity. Taking the total amount of measured carbon diffused into the media via CO₂ gas and the calculated TCO₂value, the calculated efficiency can be determined.

The second calculation (method 2) followed Standard Methods for the Examination of Water and Wastewater method 4500-D) to determine calculated IC concentrations based on the following equations:

$$[ {HCO}_{3}^{-} ] = \frac{TA - 5 \times {10}^{pH - 10}}{1 + 0.94 \times {10}^{pH - 10}} \\ [ {CO}_{3}^{2 -} ] = 0.94 [ {HCO}_{3} ] \times {10}^{pH - 10} \\ [ {CO}_{2} ] = 2 [ {HCO}_{3} ] \times {10}^{6 - pH}$$

$$Total Dissolved {CO}_{2} = [ {CO}_{2} ] + 0.44 ( 2 [ {HCO}_{3} ] + [ {CO}_{3} ] )$$

where TA=total alkalinity.

Carbonation efficiency can also be measured as follows. An exemplary calculation is shown below. MT is metric tons of biomass.

| total biomass | | | CO₂ % usage |
|---|---|---|---|
| (MT) | CO₂ usage | CO₂/MT | (carbonation efficiency) |
| 21.62869577 | 8083.75 | 373.7512 | 71% |

Inflow Slope and Outflow Slope

In order to increase the overall carbonation efficiency while keeping the back flow region to a minimum and biomass loss to a minimum, a sump contained in a pond was designed with specific ranges of inflow slopes (with about a 20 to about a 70 degree angle) and outflow slopes (with about a 5 to about a 40 degree angle). FIG. 2 describes one embodiment where the inflow slope 210 has a 30 degree angle and the outflow slope 220 has a 20 degree angle. Alternatively, the inflow slope can have an angle of about 20 degrees to about 60 degrees and the outflow slope can have an angle of about 20 to about 40 degrees. Optionally, there can be more than one inflow slope, for example, two inflow slopes and one outflow slope in the sump. In one embodiment, the inflow angle can be different that the outflow angle, as shown in FIG. 2.

The outflow slope can, for example, have two sharp corners (as shown in FIG. 2, with a 20 degree angle). Alternatively, the outflow slope can have rounded corners, both at the beginning of the slope and at the top of the slope. If both corners are rounded there will be very little back flow. Lastly, a combination of rounded and sharp corners can be used. If the corners are sharp, as in FIG. 2, a small back flow region 120 will appear.

Fluid Flow

The rate of fluid flow can be measured, for example, by meters per second or centimeters per second. One skilled in the art would be able to choose the most appropriate distance per unit time.

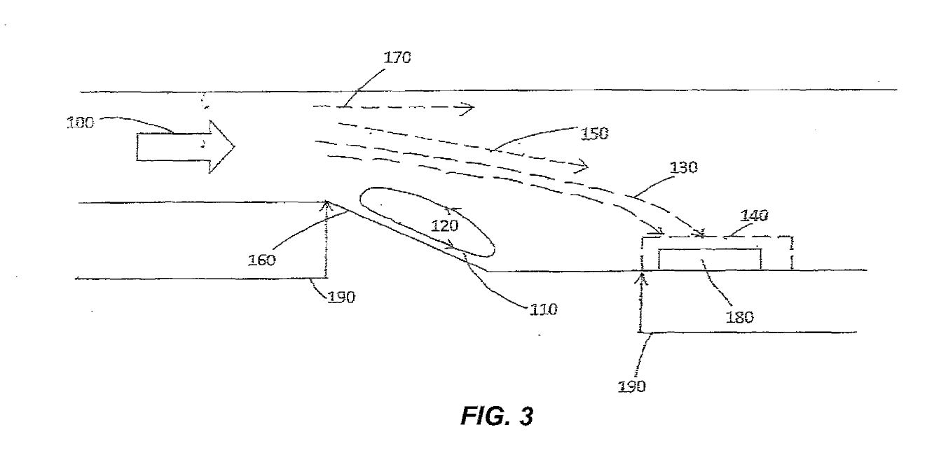

FIG. 3 shows a cultivation pond and describes the flow of the fluid over the inflow slope. A portion of the flow immediately downstream of the inflow slope 160 may reverse or otherwise proceed in a direction alternate to the bulk flow (e.g., in a direction contrary to that indicated by the arrow 100). This reverse flow, indicated by the streamline 110, may form a back flow region 120 which is represented by the elliptical shape in FIG. 3. The back flow region 120, may interface with the bulk flow (as represented by the arrow 100), in the vicinity of the streamline 130, which may be referred to as a dividing streamline and/or dividing streamline region 130. The dividing streamline 130 may interface with the pond bed in a reattachment region 140, which corresponds to the reattachment of the boundary layer 190 as shown by a vertical upward pointing arrow. As such, the reattachment region 140 may be understood as a boundary for the back flow region 120. Cultivation pond fluid may still, however, be exchanged between the bulk flow (represented by the streamlines 130 and 150) and flow in the back flow region 120 (represented by the streamline 110).

Fluid flow incident to the inflow slope may undergo boundary layer separation, as shown by the streamlines 130 and 150. Some of the fluid flow may maintain characteristics of the fluid flow upstream of the inflow slope, for example, bulk flow may be maintained in the original direction of the fluid flow 170. A diffuser 180 may be positioned in the reattachment region 140.

The back flow region as referred to in this disclosure may include, for instance a recirculation region, a closed flow loop, a separation bubble, a dynamic bubble, and/or the like. For the purposes of the present disclosure, the back flow region is considered to be distinct and separate from the bulk motion of the fluid in the cultivation pond, and the corresponding behaviors that may be observed in the bulk flow, e.g., circulation.

The back flow may further contact a surface of the inflow slope, which may induce further forward movement of the back flow. Thus, fluid flow may be captured in a closed flow loop immediately downstream of the beginning of the inflow slope. Fluid from the bulk fluid flow may be exchanged with fluid from the back flow region. Therefore, the back flow region may include reverse flow due to the separation of the boundary layer, as well as the flow that results when the back flow contacts a surface of the inflow slope and is re-oriented in the direction of the bulk flow.

Due to the presence of the inflow slope, the flow in the back flow region may become unsteady as a result of unsteady periodic vortex shedding due to the presence of the inflow slope. The back flow region may therefore result in the formation of one or more coherent structures (also termed “coherent vortices”) along the width of the channel downstream of the beginning of the inflow slope. The coherent structure may be substantially elliptical in shape (as shown in FIG. 2). A desirable back flow region of an inflow slope will measure, at its longest axis, less than approximately six times to eight times the height (y-axis) of the inflow slope angle. The back flow region may be dynamic in that it contracts, expands, shifts location in any or all directions, or any combination thereof, based on variations in the bulk flow.

The 30 to 70 degree angle of the inflow slope of the sump creates turbulence, due to the flow divergence as well as the flow separation of the liquid flowing (fluid flow) over the inflow slope. The flow separation is shown by the downward curving arrows 230 in FIG. 2. This turbulence disperses the CO₂ bubbles, shears the bubbles to break them up into smaller bubbles as well as increases the rate of mass transfer (bubbles dissolve faster in the algae culture liquid than without the turbulence). As described above, the flow separation over the inflow slope causes a flow attachment region further downstream which leads to a downward (towards the bottom of the sump) flow velocity component that aids in preventing bubbles from reaching the surface of the liquid.

The angle of the inflow slope creates turbulence that is needed to prevent the settling or sedimentation of the organism, for example, algae, to the sump bottom. A steep inflow slope with an angle of 90 degrees (also known as a “backward-facing” step) results in a high degree of turbulence but also a large undesirable back flow region is generated in the fluid flow downstream of the inflow slope resulting in a large toss of algal biomass. In addition, bacteria or other invaders that may be harmful to the algal culture can live in the back flow region. Dirt or debris may also be found in the back flow region. In contrast, a smaller slope, such as that with about a 30 to about a 70 degree angle results in less turbulence, but still enough to prevent some settling of the algae, and results in a smaller back flow region, more algal biomass, and less change of invaders that may be harmful to the algal culture. The larger the back flow region present in the sump the less surface production area is present. A desirable back flow region of an inflow slope is shown as an elliptical shape in FIG. 2.

The presence of a back flow region may prevent the formation of undesirable concentration gradients in the cultivation pond. Although, as described above, too big of a back flow region will decrease the amount of algal biomass that is recovered. In addition, the turbulence from the back flow region can aid in the uptake of carbon dioxide in the culture. Therefore careful thought has to be applied as to how big a back flow region is desired.

The longest axis of a back flow region of a 90 degree backward-facing step can be, for example, about 6-8 times the height of the step which is undesirable due to the large loss of recoverable biomass. The flow properties of a backward-facing step have been well studied (for example, as described in Armaly, B. F., et al., J. Fluid Mech. (1983)127:473-496). The back flow region in the literature has been called the “back flow length”, the “separated region length”, and the “reattachment length”.

Calculation of Longest Axis of Back Flow Region

The size of the longest axis of the back flow region is a function of for example, the flow speed, depreciation depth, surface roughness of the pond bottom, upstream flow conditions such as Reynolds number and boundary layer thickness, sharpness or curvature of the edges (quality of the edges) and the slope of the inflow angle. The length of the longest axis can be measured or simulated using sophisticated software such as ANSYS CFX or ANSYS Fluent (ANSYS, USA), among others.

ANSYS CFX software is a high-performance, general purpose fluid dynamics program that can be applied to solve wide-ranging fluid flow problems. At the heart of ANSYS CFX is its solver technology; the highly parallelized solver is the foundation for a wide choice of physical models to capture virtually any type of phenomena related to fluid flow. ANSYS Fluent software contains the broad physical modeling capabilities needed to model flow, turbulence, heat transfer, and reactions for industrial applications. Both ANSYS CFX and ANSYS Fluent are well known software programs that one skilled in the art would be familiar with. It would be well within the ability of one skilled in the art to use and generate the length of the longest axis of a back flow region using the disclosed software.

Exemplary technical resources available to one skilled in the art are: ANSYS CFX Tutorials, Release 13.0, November 2010, ANSYS, Inc.; Computational Fluid Dynamics for Fluids Engineering Design, ANSYS CTX Student User Manual, Version 11, Gordon D. Stubley, Department of Mechanical Engineering, University of Waterloo (Jan. 30, 2008); and ANSYS FLUENT Tutorial Guide, Release 14.0, November 2011Ansys Inc.

The data provided below regarding the longest axis measurements are estimates based on several of these types of sophisticated calculations.

TABLE A

| | | Longest | Percentage of | Percentage of |
|---|---|---|---|---|
| | | axis of | total surface | total surface |
| | | back flow | area of a 50 | area of a 200 |
| Angle of | Depreci- | region of | foot long raceway | foot long raceway |
| inflow | ation | inflow | pond that is the | pond that is the |
| slope | depth | slope | back flow region | back flow region |
| 30 degrees | 21 inches | Approx- | 2.5% to 3.3% | 0.6% to 0.8% |
| | | imately | | |
| | | 30-40 | | |
| | | inches | | |
| 45 degrees | 21 inches | Approx- | 4.1% to 6.6% | 1.0% to 1.6% |
| | | imately | | |
| | | 50-80 | | |
| | | inches | | |
| 60 degrees | 21 inches | Approx- | 7% to 9.3% | 1.7% to 2.3% |
| | | imately | | |
| | | 84-112 | | |
| | | inches | | |
| 90 degrees | 21 inches | Approx- | 10.5% to 14% | 2.6% to 3.5% |
| | | imately | | |
| | | 126-168 | | |
| | | inches | | |

Calculating the Percentage of Total Surface Area of a 50 Foot Long Raceway Pond that is the Back Flow Region of the Inflow Slope.

For the calculations in Table A, the sump spans the pond width, so the pond width is equal to the sump width. Also for the above-cited calculations, the back flow region covers the entire sump width. The sump may not span the entire width of the pond. If this is the case, one skilled in the art would still be able to calculate the percentage of total surface area of a raceway pond that is the back flow region.

Example: for a pond of 50 ft long and 4 ft wide if the longest axis of the back flow region of the inflow slope is 40 inches long (3.3 ft long) the percentage of total surface area of the dead zone (back flow region) is (3.3×4)(2×50×4)=0.033 or 3.3% as shown in the above Table A. The factor 2 is used here since in a raceway pond there are 2 channels each 50 ft long. Another example is, if the channel width is 6 ft then the calculation becomes: (3.3×6)/(2×50×6)=0.033 or 3.3%.

Example: for a pond of 50 ft long and 4 ft wide if the longest axis of the back flow region of the inflow slope is 80 inches long (6.6 ft long) the percentage of total surface area of the dead zone (back flow region) is (6.6×4)/(2×50×4)=0.066 or 6.6% as shown in the above Table A. The factor 2 is used here since in a raceway pond there are 2 channels each 50 ft long. Another example is, if the channel width is 6 ft then the calculation becomes: (6.6×6)/(2×50×6)=0.066 or 6.6%.

When calculating the percentage of total surface area of a pond that is the back flow region, the area of the two “turns” in the ponds (as shown in FIG. 4), located at each end, is not considered. An exemplary raceway pond, with the length of each of the two channels being shown as 330 and the width of each of the two channels being shown as 320, is illustrated in FIG. 4.

Calculating the Total Surface Area of a Raceway Pond.

When calculating the total surface area of a pond, the area of the two “turns” in the ponds (as shown in FIG. 4), one located at each end, is not considered. An exemplary raceway pond, with the length of each of the two channels being shown as 330 and the width of each of the two channels being shown as 320, is illustrated in FIG. 4.

Example: for a pond where the length of the pond is 200 feet, the width of the pond is 16 feet, and the number of raceway channels is 2, then 2×200×16=6,400 square feet or approximately 600 m2. One skilled in the art would be able to calculate the total surface area of various ponds of different length, widths, and configurations (for example, “race way” or serpentine).

Desirable Volume of Back Flow Region of the Inflow Slope

As described above, a back flow region should not be too large, in order to minimize the loss of biomass that can be recovered from the pond, while stilling maintaining turbulence. It may be desirable to have, for example, no more than about 25% of the total volume of the sump be the back flow region of the inflow slope. Provided below in Table B are examples of desirable percent volumes of back flow regions (VR) for inflow slopes as compared to the total volume of the sump for several different size sumps. The width of the sump (column 3 from the left) is measured at the bottom of the pond. Non-limiting examples of percent volumes of back flow regions of the inflow slope as compared to the total volume of the sump are from 0 to about 75%, 0 to about 50%, from about 0.52 to about 25.0%, from about 18.75% to about 25.0%, from about 13.82-18.42%, or from about 9.05 to 12.07%. The back flow region of the inflow slope can also have a volume of less than 45%, less than 40%, less than 35%, less than 30%, less than 25%, less than 20%, less than 15%, less than 10%, or less than 5% of the total volume of the sump.

Volume Ratio (VR) can be calculated as provided below.

$$VR = 2 \times \frac{( C \times h )}{( L + {L}_{1} )} \times 100$$

“L” is the length of the bottom of the sump (as shown in FIG. 2 as 270).

“L₁” is the length from the beginning of the inflow slope to the end of the outflow slope (as shown in FIG. 2 as 280).

C is about 6 or about 8, or any number in between 6 and 8.

“h” is height (as described below).

“d” is depreciation depth as is shown in FIG. 2 as 290

Exemplary desirable volumes of back flow regions of the inflow slope as compared to the total volume of the sump are shown in Table B in the column labeled “% of total vol. of sump=volume ratio (VR)”. The range of VR values in Table B represent C=6 to C=8. C=is the longest axis of a back flow region as described in the literature (for example, as described in Armaly, B. F., et al., J. Fluid Mech. (1983) 127:473-496) which is 6 to 8 times the height of a 90 degree backward-facing step.

For an inflow slope with a 30 degree angle, in our test pond it was observed that the flow separates after it travel approximately 75% of the distance of the diverging wall (as shown in FIG. 2 as 310). Therefore, height (h) is equal to ¼ (25%) of the sump depreciation depth.

For other inflow angles (for example, such as 35 degrees, 40 degrees, 45 degrees, 50 degrees, 55 degrees, 60 degrees, 65 degrees or 70 degrees) one skilled in the art can experimentally determine the point of flow separation, for example by creating a test pond with a desired angle, and observing the distance in which the flow separates.

The distance in which the flow separates after it travels over the diverging wall is dependent on, for example, certain flow characteristics, such as flow velocity, Reynold's number, turbulence level, and upstream boundary layer thickness, and also on the surface roughness of the pond floor.

Exemplary flow separation distances for inflow angels of 20, 25, 30, 35, 40, 45, 50, 55, 60, 65 and 70 degrees are provided below. The number in “( )” is the number to multiply by the depreciation depth “d” (in inches), and divide by 12 to get “h” in feet; “h” will be used in the above equation in order to calculate VR.

For a 20 degree inflow slope: the flow separates after it travels about 85% of the distance of the diverging wall; (about 0.15). For a 25 degree inflow slope: the flow separates after it travels about 80% of the distance of the diverging wall; (about 0.2). For a 30 degree inflow slope: the flow separates after it travels about 75% of the distance of the diverging wall; (0.25). For a 35 degree inflow slope: the flow separates after it travels about 67% of the distance of the diverging wall; (about 0.33). For a 40 degree inflow slope: the flow separates after it travels about 58% of the distance of the diverging wall; (about 0.42). For a 45 degree inflow slope: the flow separates after it travels about 50% of the distance of the diverging wall; (0.5). For a 50 degree inflow slope: the flow separates after it travels about 42% of the distance of the diverging wall; (about 0.58). For a 55 degree inflow slope: the flow separates after it travels about 33% of the distance of the diverging wall; (about 0.75). For a 60 degree inflow slope: the flow separates after it travels about 25% of the distance of the diverging wall; (0.75). For a 65 degree inflow slope: the flow separates after it travels about 22% of the distance of the diverging wall; (about 0.78). For a 70 degree inflow slope: the flow separates after it travels about 20% of the distance of the diverging wall; (about 0.8).

Each value in the formula presented above is converted from inches to feet. For example, the depreciation depth of 21 inches is multiplied by 0.25 (¼ of the sump depreciation depth) and then divided by 12 so that h=0.4375 feet. The calculation for “h” is provided below.

$$h = \frac{21 ( 0.25 )}{12}$$

So, for example, for a sump that is represented by the first line of Table B the following VR values are calculated for C=6 and C=8.

$$VR = 2 \times \frac{( 6 \times ( 0.4375 ) )}{( 10 + 18 )} \times 100 = 18.75 \%$$

$$VR = 2 \times \frac{( 8 \times ( 0.4375 ) )}{( 10 + 18 )} \times 100 = 25.0 \%$$

Rows 1, 3, and 5 represent dimensions of a sump located within a 0.33 acre, 1.1 acre, and 2.2 acre “raceway” cultivation pond, respectively; the raceway ponds contain two channels. An exemplary raceway pond is shown in FIG. 4.

TABLE B

| | | | | | % of total | | |
|---|---|---|---|---|---|---|---|
| length | length | width | inflow | | vol. of sump = | sump | ranges |
| of sump | of sump | of sump | slope | depreciation | volume ratio | volume in | of VR in |
| (L) | (L₁) | (W) | angle | depth (d) | (VR) | cubic feet | cubic feet |
| 10 ft | 18 ft | 8 ft 8 | 30 degrees | 21 inches | 18.75-25.0% | 212.17 | 39.78-53.04 |
| | | inches | | | | | |
| 12 ft | 20 ft | 16 ft | 30 degrees | 21 inches | 16.40-21.88% | 448 | 73.48-98.02 |
| 15 ft | 23 ft | 26 ft 2 | 30 degrees | 21 inches | 13.82-18.42% | 870 | 120.23-160.254 |
| | | inches | | | | | |
| 20 ft | 28 ft | 15 ft | 30 degrees | 21 inches | 10.94-14.58% | 630 | 68.92-91.85 |
| 25 ft | 33 ft | 26 ft 2 | 30 degrees | 21 inches | 9.05-12.07% | 1327.92 | 120.18-160.28 |
| | | inches | | | | | |
| 30 ft | 38 ft | 15 ft | 30 degrees | 21 inches | 7.72-10.29% | 892.5 | 68.9-91.84 |
| 50 ft | 58 ft | 15 ft | 30 degrees | 21 inches | 4.86-6.48% | 1417.5 | 68.9-91.85 |
| 100 ft | 108 ft | 16 ft | 30 degrees | 21 inches | 2.52-3.37% | 2912 | 73.38-98.13 |
| 200 ft | 208 ft | 32 ft | 30 degrees | 25 inches | 1.53-2.04% | 13,600 | 208-277.44 |
| 500 ft | 508 ft | 32 ft | 30 degrees | 21 inches | 0.52-0.69% | 28,224 | 146.76-194.75 |
| 500 ft | 508 ft | 32 ft | 30 degrees | 25 inches | 0.62-0.83% | 33,546 | 207.99-278.43 |

Calculating the Volume of the Sump

$$Sump Volume = \frac{( L + {L}_{1} )}{( 2 )} \times d \times W$$

W is the width of the sump. “d” is depreciation depth. In Table B, sump volume is calculated in cubic feet, so each value in the formula presented above is converted from inches to feet. Volume can be, for example, in cubic feet, cubic inches, gallons or any other measure of unit known to one skilled in the art. Exemplary sump volumes are shown in Table B in the column labeled “sump volume in cubic feet”.

Sump Depth, Length, Width and Shape

The depth of the sump can be measured in at least two different ways. The distance from the liquid surface to the bottom of the sump is the water depth and the distance from the bottom of the sump to the top of the inflow or outflow slope is called the depreciation depth. The water depth is variable. For example, the water depth can be about 6 to about 60 inches, or about 8 to about 40 inches. Other non-limiting examples of water depth are: about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, about 24 inches, about 25 inches, about 26 inches, about 27 inches, about 28 inches, about 29 inches, about 30 inches, about 31 inches, about 32 inches, about 33 inches, about 34 inches, about 35 inches, about 36 inches, about 37 inches, about 38 inches, about 39 inches, about 40 inches, about 41 inches, about 42 inches, about 43 inches, about 44 inches, about 45 inches, about 46 inches, about 47 inches, about 48 inches, about 49 inches, about 50 inches, about 51 inches, about 52 inches, about 53 inches, about 54 inches, about 55 inches, about 56 inches, about 57 inches, about 58 inches, about 59 inches, or about 60 inches. The depreciation depth can be from about 5 inches to about 50 inches, or from about 10 to about 30 inches. Other non-limiting examples of depreciation depth are: about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49 or about 50 inches. Alternatively, the depreciation depth can be about 21 inches.

The length of the sump is measured from the end of the inflow slope to the beginning of the outflow slope. The length of the sump can be from about 2 feet to about 1000 feet, from about 10 feet to about 500 ft, or from about 20 to about 200 feet. Other non-limiting examples of a length of the sump are: about 2 feet, about 4 feet, about 6 feet, about 8 feet, about 10 feet, about 12 feet, about 14 feet, about 16 feet, about 18 feet, about 20 feet, about 22 feet, about 24 feet, about 26 feet, about 28 feet, about 30 feet, about 32 feet, about 34 feet, about 36 feet, about 38 feet, about 40 feet, about 42 feet, about 44 feet, about 46 feet, about 48 feet, about 50 feet, about 52 feet, about 54 feet, about 56 feet, about 58 feet, about 60 feet, about 62 feet, about 64 feet, about 66 feet, about 68 feet, about 70 feet, about 72 feet, about 74 feet, about 76 feet, about 78 feet, about 80 feet, about 85 feet, about 90 feet, about 100 feet, about 110 feet, about 120 feet, about 130 feet, about 140 feet, about 150 feet, about 160 feet, about 170 feet, about 180 feet, about 190 feet, or about 200 feet. For example, as shown in FIG. 2, the length of the sump is 144 inches or 12 feet. Alternatively, the length of the sump is about 10 feet 10 inches, about 15 feet, or about 25 feet,

The width of the sump can be the width of the cultivation channel. Alternatively, the width of the sump can be, for example, from about 1 foot to about 500 feet wide, from about 10 feet to about 250 feet wide, or from about 20 feet to about 100 feet wide. Other non-limiting examples of a width of the sump are: about 5 feet, about 10 feet, about 15 feet, about 20 feet, about 25 feet, about 30 feet, about 35 feet, about 40 feet, about 45 feet, about 50 feet, about 55 feet, about 60 feet, about 65 feet, about 70 feet, about 75 feet, about 80 feet, about 85 feet, about 90 feet, about 95 feet, about 100 feet, about 110 feet, about 120 feet, about 130 feet, about 140 feet, about 150 feet, about 160 feet, about 170 feet, about 180 feet, about 190 feet, or about 200 feet. More non-limiting examples of a width of the sump are: about 4 feet, about 6 feet, about 8 feet, about 10 feet, about 12 feet, about 14 feet, about 16 feet, about 18 feet, about 20 feet, about 22 feet, about 24 feet, about 26 feet, about 28 feet, about 30 feet, about 32 feet, about 34 feet, about 36 feet, about 38 feet, or about 40 feet. The sump can have a width of about 8 feet 8 inches, about 15 feet, or about 26 feet 2 inches. The width of the sump is measured mid way up or near the top of the side wall (along the length) of the sump. The width of the sump can also be measured at the bottom of the side wall (along the length) of the sump.

Cultivation ponds can be in the form of raceways (for example, Oswald raceway ponds) or any other shapes such as serpentine or circular.

A pond can have a width, at any given point, of 1 foot to 10,000 feet. A pond can have a width, at any given point of 100-500 feet, 500-1000 feet, or 1000-10,000 feet. A cultivation pond can be, for example, an open field. A pond can have any shape, for example, circular, oval, square, rectangular, a non-uniform shape (for example, an open field or pond) or any combination of above shapes.

For the raceway or serpentine ponds, the length of the pond channels can be from about 20 feet to about 10 miles or from about 200 feet to about 3 miles. Other non-limiting examples of a length of a pond are: about 200 feet long, about 250 feet long, about 300 feet long, about 350 feet long, about 400 feet long, about 450 feet long, about 500 feet long, about 550 feet long, about 600 feet long, about 650 feet long, about 700 feet long, about 750 feet long, about 800 feet long, about 850 feet long, about 900 feet long, about 950 feet long, about 1000 feet long, about 1500 feet long, about 2000 feet long, about 2500 feet long, about 3000 feet long, about 3500 feet long, about 4000 feet long, about 4500 feet long, about 5000 feet long, about 5500 feet long, about 6000 feet long, about 6500 feet long, about 7000 feet long, about 7500 feet long, about 8000 feet long, about 8500 feet long, about 9000 feet long, about 9500 feet long, about 10,000 feet long, about 10,500 feet long, or about 11,000 feet long. The length of the pond can be about 0.1 mile long, about 0.2 miles long, about 0.3 miles long, about 0.4 miles long, about 0.5 miles long, about 0.6 miles long, about 0.7 miles about long, about 0.8 miles long, about 0.9 miles long, about 1.0 miles long, about 1.1 miles long, about 1.2 miles long, about 1.3 miles long, about 1.4 miles long, about 1.5 miles long, about 1.6 miles long, about 1.7 miles long, about 1.8 miles long, about 1.9 miles long, or about 2.0 miles long. Alternatively, the length of the pond can be about 623 feet or about 1279 feet, from about 600 feet to about 650 feet, or from about 1200 feet to about 1300 feet.

The area of the pond can be from about 36 square feet to about 500 acres. The area of the cultivation pond can be, for example, about 25 square feet to about 500 acres, or from about 50 square feet to about 100 acres. The area of the pond can be, for example, about 1 acre, about 1.1 acre, about 1.2 acres, about 1.3 acres, about 1.4 acres, about 1.5 acres, about 2 acres, about 3 acres, about 4 acres, or about 5 acres. For example, the area of the pond can be about 6,400 square feet.

Exemplary Cultivation Ponds

FIG. 10 and FIG. 11 show two exemplary raceway cultivation ponds. The width 240 of each of the two channels in both FIG. 10 and FIG. 11 is about 35 feet. In FIG. 10 the length 250 of the pond is about 623 feet long. In FIG. 11 the length 260 of the pond is about 1279 feet. When calculating the width and length of each of the channels the two “curves” located at each end of the cultivation pond were not taken into consideration. As described above, the location, number, and design of the diffusers within the cultivation pond and/or sump can vary. In addition the location of a fluid distribution system (described below), if one such system is needed, can also vary.

Shown in Table C are other non-limiting exemplary dimensions and areas of several raceway cultivation ponds and sumps.

| | | | | | width of a | width of a |
|---|---|---|---|---|---|---|
| | length | length | | length of | pond channel | pond channel |
| area of | of a sump | of sump | width | each pond | at the bottom | at a water depth |
| pond | (L) | (L₁) | of a sump | channel | of the pond | of 10 inches |
| 0.33 acres | 10 feet | 18 ft | 8 feet 8 | 218 ft | 16 ft | 21 ft |
| | | | inches | | | |
| 1.1 acre | 15 feet | 23 ft | 26 feet 2 | 636 ft | 32 ft | 38.3 ft |
| | | | inches | | | |
| 2.2 acres | 25 feet | 33 ft | 26 feet 2 | 1288 ft | 32 ft | 38.3 ft |
| | | | inches | | | |

There can be more than one sump per cultivation pond, for example, there can be multiple sumps located at scveral locations throughout the pond. For example, if a pond is very long, algae will consume CO₂ as the algae travels along the pond, therefore sumps can be built at the positions where it is calculated/estimated that the algae will run out of CO₂. The number of sumps may be dependent on the organism being grown and the size of the cultivation pond. For example, a Nannochloropsis strain may need a pH of about 8.0 for optimal growth, and may be able to travel around a raceway pond for an hour before needing an injection of CO₂ into the solution it is growing in. The addition of CO₂ can reduce the pH and make the solution that the organism is growing in more acidic. Alternatively, a Spirulina strain may need a pH of about 10-11 for optimal growth, and may be able to travel around the raceway pond for up to four hours before receiving an injection of CO₂. One skilled in the art could determine the optimal pH that a strain can be grown in by, for example, by growing the strain in several different pHs and monitoring the growth rate at the different pHs. The strain can also be tested to see how often it needs to have more CO₂ injected into the solution in order to maintain an optimal growth rate and optimal pH.

The following equation can be used to calculate the distance between the end of the outflow slope of a first sump and the beginning of the inflow slope of a second sump. It should be noted that there does not need to be more than one sump per cultivation pond. This calculation assumes that the flow velocity of the liquid is constant while circulating around the pond. However, there may be variation, for example, the flow velocity in the channels may be different than the flow velocity in the turns of the pond. The x value is provided as a starting point, then one skilled in the art could do further calculations taking into consideration such things, for example, as the flow velocity in the turns, to determine the exact distance needed. The example provided below, it is understood that the sump provides the source of CO₂ and that the CO₂ doesn't come from a source located between any two of the sumps present in the pond. The flow velocity can be measured by various flow meters known to one skilled in the art, such as a Venturi Tube flow meter.

x=Ut

x=length in between the end of the outflow slope of a first sump and the beginning of the inflow slope of a second sump

U=flow velocity (for example, in centimeters per second (cm/s))

t=time interval (for example, seconds) before more is CO₂is needed to be injected into the solution, determined as described above

Example: a Strain that Can Go for Four Hours Before Needing an Injection of CO₂

4 hours=14,400 seconds

U=15 cm/s

x=Ut

x=15 cm/s×14,400 seconds

x=216,000 cm or 2160 meters

Fluid Distribution System

The sump can optionally be located from about 1 foot to about 500 feet, from about 2 ft to about 200 ft downstream or from about 5 ft to about 50 ft downstream of a fluid distribution system, such as a paddlewheel, a jet circulation system (for example, as described in U.S. Ser. No. 12/485,862), a direct pump, an Archimedes screw, any newly developed device that can agitate the flow of liquid in the pond, or any combination of the above. The sump can be located, for example, about 1 foot, about 2 feet, about 3 feet, about 4 feet, about 5 feet, about 6 feet, about 7 feet, about 8 feet, about 9 feet, about 10 feet, about 11 feet, about 12 feet, about 13 feet, about 14 feet, about 15 feet, about 16 feet, about 17 feet, about 18 feet, about 19 feet, about 20 feet, about 22 feet, about 24 feet, about 26 feet, about 28 feet, or about 30 feet, down stream of a fluid distribution system. Alternatively, the sump can be located about 10 feet downstream of a fluid distribution system. The paddle wheel can be placed about 2 feet, about 4 feet, about 6 feet, about 8 feet, about 10 feet, about 12 feet, about 14 feet, about 16 feet, about 18 feet, about 20 feet, about 22 feet, about 24 feet, about 26 feet, about 28 feet, or about 30 feet upstream of the inflow angle. The fluid distribution system serves to create background turbulence. If the inflow slope has an angle of 90 degrees (a backward-facing step), a fluid distribution system is not needed because the 90 degree angle results in severe turbulence.

A fluid distribution system may include a series of paddle wheels, which can be used to initiate fluid flow in the cultivation pond. Similarly, a jet circulation system as referred to above may be used, and nozzles associated with the jet circulation system may be submerged in the cultivation pond fluid.

Pond

The pond can be, for example, an open-air raceway pond design (as shown in FIG. 4). The location of an exemplary sump, paddle wheel, and diffuser are shown in FIG. 4. One skilled in the art will recognize that the disclosure herein may be adapted to other pond designs, for instance single channels, flumes, and the like. The cultivation pond my be filled with cultivation pond fluid to any desirable depth. Cultivation pond fluid can be any liquid, for example, culture media. Culture media can be supplemented, for example, with nutrients.

Construction of Pond and Sump

The pond and/or sump can be made using, for example, any concrete, a mixture of concretes, or a mixture of a concrete with any other substance know to one skilled in the art. The pond and/or sump can also be made using a plastic or another substance that is capable of being shaped into a desired form and able to contain a liquid. The pond and/or sump can also be dug directly into existing dirt or ground.

The pond and/or sump can, optionally, can be lined with a lining such as Hypalon (chlorosulfonated polyethylene (CSPE) synthetic rubber (CSM) (DuPont Performance Elastomers, US; Tosoh Coporation, Japan)). CSM is noted for its resistance to chemicals, temperature extremes, and ultraviolet light.

Organisms

The selection of an appropriate organism is deemed to be within the scope of those skilled in the art. Examples of organisms that can be grown in the cultivation pond and sump include non-vascular photosynthetic organisms. The organism can be prokaryotic or cukaryotic. The organism can be unicellular or multicellular. A non-vascular photosynthetic organism is one that naturally photosynthesizes (e.g., an alga) or that is genetically engineered or otherwise modified to be photosynthetic. As used herein, the term “non-vascular photosynthetic organism,” refers to any macroscopic or microscopic organism, including, but not limited to, algae, photosynthetic bacteria (including cyanobacteria), which do not have a vascular system such as that found in vascular plants. In some instances the organism is a cyanobacterium. In some instances, the organism is algae (e.g., macroalgae or microalgae). Examples of non-vascular photosynthetic organisms include bryophtyes, such as marchantiophytes or anthocerotophytes.

The organism can be prokaryotic. Examples of some prokaryotic organisms of the present disclosure include, but are not limited to, cyanobacteria (e,g., Synechococcus, Synechocystis, Athrospira, Spirulina, Leptolyngbya, Lyngbya, Gleoecapsa, Oscillatoria, and, Pseudoanabaena).

Examples of cyanobacteria that can be used in the embodiments disclosed herein include Synechococcus sp., Spirulina sp., Synechocystis sp. Athrospira sp., Prochlorococcus sp., Chroococcus sp., Gleoccapsa sp., Aphanocapsa sp., Aphanothece sp., Merismopedia sp., Microcystis sp., Coelosphaerium sp., Prochlorothrix sp., Oscillatoria sp., Trichodesmium sp., Microcoleus sp., Chroococcidiopisis sp., Anabaena sp., Aphanizomenon sp., Cylindrospermopsis sp., Cylindrospermum sp., Tolypothrix sp., Leptolyngbya sp., Lyngbya sp., or Scytonema sp.

In some embodiments, the organism is eukaryotic (e.g., green algae, red algae, brown algae). In some embodiments, the alga is a green alga, for example, a Chlorophyccan. The algae can be unicellular or multicellular. Suitable eukaryotic cells include, but are not limited to, plant cells and algal cells. In some embodiments, microalgae, such as for example, a Chlamydomonas, Volvacales, Dunaliella, Nannochloropsis, Desmodesmus, Scenedesmus, Chlorella, Volvox, Arthrospira, Spirulina, Botryococcus, Desmid, Vannochloropsis, or Hematococcus species, can be used in the disclosed methods, cultivation pond and sump. In other embodiments, the organism is Chlamydomonas reinhardtii, Dunaliella salina, Haematococcus pluvialis, Nannochloropsis oceania, Nannochloropsis salina, Scenedesmus dimorphus, Spirulina maximus, Arthrospira fusiformis, Dunaliella viridis, Nannochloropsis oculata, Desmodesmus maximus, or Dunaliella tertiolecta. In some instances the organism is a rhodophyte, chlorophyte, heterokontophyte, tribophyte, glaucophyte, chlorarachniophyte, euglenoid, haptophyte, cryptomonad, dinoflagellum, or phytoplankton.

Some of the organisms useful in the disclosed embodiments are, for example, are extremophiles, such as hyperthermophiles, psychrophiles, psychrotrophs, chrotrophs, halophiles, barophiles and acidophiles. Some of the organisms which may be used to practice the present disclosure are halophilic (e.g., Dunaliella salina, D. viridis, or D. tertiolecta). For example, D. salina can grow in ocean water and salt lakes (for example, salinity from 30-300 parts per thousand) and high salinity media (e.g., artificial seawater medium, seawater nutrient agar, brackish water medium, and seawater medium). In some embodiments of the disclosure, an organism of the present disclosure is grown or cultivated in a liquid medium (environment) which is, for example, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3 molar or higher concentrations of sodium chloride. One of skill in the art will recognize that other salts (sodium salts, calcium salts, potassium salts, or other salts) may also be present in the liquid medium.

Culturing of Organisms

Organisms can be grown or cultivated in a liquid medium. A liquid medium can be water, or any other liquid. Non-limiting liquid medium are seawater medium, minimal medium, culture media, high salt medium, synthetic media, enriched media, seawater media, high salt media, a high pH media (for example, above pH 9), or any other media or medium known to one skilled in the art as long as it is a liquid.

Organisms can be grown or cultivated in a defined minimal medium (for example, high salt medium (HSM), modified artificial sea water medium (MASM), or F/2 medium) with light as the sole energy source. In other instances, the organism can be grown in a medium (for example, tris acetate phosphate (TAP) medium), and supplemented with an organic carbon source. In addition, a demonstration-scale media designed to mimic and control the amount of total dissolved solids during steady-state growth of the organism can be used in the cultivation pond.

Organisms, such as algae, can grow naturally in fresh water or marine water. Culture media for freshwater algae can be, for example, synthetic media or enriched media. Various culture media have been developed and used for the isolation and cultivation of fresh water algae and are described in Watanabe, M. W. (2005). Freshwater Culture Media, In R. A. Andersen (Ed.), Algal Culturing Techniques (pp. 13-20). Elsevier Academic Press. Culture media for marine algae can be, for example, artificial seawater media or natural seawater media. Guidelines for the preparation of media for marine algae are described in Harrison, P. J. and Berges, J. A. (2005). Marine Culture Media. In R. A. Andersen (Ed.), Algal Culturing Techniques (pp. 21-33). Elsevier Academic Press. Culturing techniques for algae are well know to one of skill in the art and are described, for example, in Freshwater Culture Media. In R. A. Andersen (Ed.), Algal Culturing Techniques. Elsevier Academic Press.

Organisms may be grown or cultivated in outdoor open water, such as ponds, the ocean, seas, rivers, waterbeds, marshes, shallow pools, lakes, aqueducts, and reservoirs. Because photosynthetic organisms, for example, algae, require sunlight, CO₂ and water for growth, they can be cultivated in, for example, in an outdoor race way pond (e.g. cultivation pond). Another approach to growing an organism is to use a semi-closed system, such as covering the race way pond or pool with a structure, for example, a “greenhouse-type” structure. One or more species of organisms can be grown in the same space (for example, a race way pond), in other words, a monoculture or a mixed culture can be grown in the cultivation pond or sump of the disclosure.

In a “race way” pond, the organism, water, and nutrients circulate around a race way or “race track.” A paddlewheel or paddlewheels provide constant motion to the liquid in the race track, allowing for the organism to be circulated back to the surface of the liquid at a chosen frequency. A paddlewheel also provides a source of agitation and oxygenate the system. These raceway ponds can be enclosed, for example, in a building or a greenhouse, or can be located outdoors. The raceway ponds can be operated in a continuous manner, with, for example, CO₂and nutrients being constantly fed to the ponds, while water containing the organism is removed at the other end. If the raceway pond is placed outdoors, there are several different ways to address the invasion of an unwanted organism. For example, the pH or salinity of the liquid in which the desired organism is in can be such that the invading organism either slows down its growth or dies. Also, chemicals can be added to the liquid, such as bleach, or a pesticide can be added to the liquid, such as glyphosate. In addition, the organism of interest can be genetically modified or evolved such that it is better suited to survive in the liquid environment. Any one or more of the above strategies can be used to address the invasion of an unwanted organism.

Organisms can also be grown near ethanol production plants or other facilities or regions (e.g., cities and highways) generating CO₂.

The organism of interest, grown in any of the systems described herein, can be, for example, continually harvested, or harvested one batch at a time.

Nutrients that can be added to the liquid, contained in the cultivation pond liquid described herein, include, for example, nitrogen (in the form of NO₃⁻or NH₄⁺), phosphorus, and trace metals (Fe, Mg, K, Cu, Co, Cu, Mo, Zn, V, and B). The nutrients can come, for example, in a solid form or in a liquid form. If the nutrients are in a solid form they can be mixed with, for example, fresh or salt water prior to being delivered to the liquid containing the organism.

Organisms can be grown in culture, for example large scale culture, wherein large scale culture refers to the growth of organisms in a volume of liquid that is greater than about 50 liters, greater than about 200 liters, greater than about 500 liters, or greater than about 1000 liters. Large scale cultures can also refer to the growth of organisms in a volume of liquid that is greater than 50,000 liters, greater than 100,000 liters, or greater than 150,000 liters. Large scale cultures of organism can be grown in a volume of liquid of about 400 liters to about 500,000 liters.

Large scale cultures can be grown in, for example, ponds, containers, vessels, or other areas, where the pond, container, vessel, or area that contains the culture is for example, at least 5 square meters, at least 10 square meters, at least 200 square meters, at least 500 square meters, at least 1,500 square meters, at least 2,500 square meters, or at least 5,000 square meters, in area, or greater.

Chlamydomonas sp., Nannochloropsis sp., Scenedesmus sp., Desmodesmus sp., and Chlorelia sp. are exemplary algae that can be cultured as described herein and can grow under a wide array of conditions.

Optimal growth of organisms occurs usually at a temperature of about 20° C. to about 25° C., although some organisms can still grow at a temperature of up to about 35° C. An exemplary growth rate may yield, for example, a two to twenty fold increase in cells per day, depending on the growth conditions. In addition, doubling times for organisms can be, for example, 5 hours to 30 hours.

One source of energy, other than the sun, is fluorescent light that can be placed, for example, at a distance of about 1 inch to about two feet from the organism. Examples of types of fluorescent lights includes, for example, cool white and daylight. Bubbling with air or CO₂ improves the growth rate of the organism. Bubbling with CO₂ can be, for example, at 1% to 5% CO₂.

The following examples are intended to provide illustrations of the application of the present disclosure. The following examples are not intended to completely define or otherwise limit the scope of the disclosure. One of skill in the art will appreciate that many other methods known in the art may be substituted in lieu of the ones specifically described or referenced herein.

### EXAMPLES

### Example 1

### Carbonation Efficiency in the Absence of a Sump

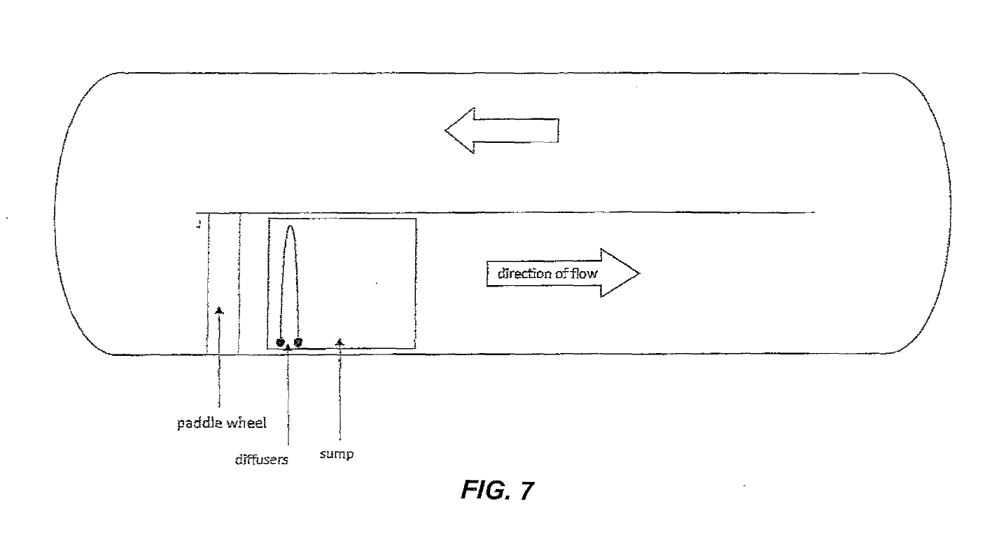

In EXAMPLE 1, a 6,400 square foot raceway pond was used to measure carbonation efficiency in the absence of a sump. Diffusers were set up as shown in FIG. 7. FIG. 7 shows one 25 foot diffuser folder back upon itself located along the width of the pond. Municipal water with algae was tested.

Diffusers

Aquatech (Aquatech Environmental System Ltd., Canada) diffuser hoses were secured at the bottom of the pond using a flat metal frame and were connected to a CO₂ feeding line. Aquatech diffuser hoses are made of thermal plastic rubber. The thickness of the wall of the Aquatech diffuser is 1/16 inch. The inside diameter of the Aquatech diffuser hose is ½ inch.

Pressure Drop Measurement

A standard pressure gauge was installed before and after the location of the diffuser hoses to measure the pressure drop along the diffuser hoses at different CO₂ flow rates. In FIG. 7 the two circles at the ends of the U-shaped diffuser hose are pressure gauges.

CO₂ Mass Flow Meter and Programmable Logic Controller (PLC) Set Up

A mobile CO₂ PLC cabinet was located near the 6,400 square ft raceway pond, connecting the diffuser and the CO₂ feed line. The cabinet is composed of a CO₂ digital mass flow meter, a pressure regulator, and a PLC. 100% CO₂ gas was injected into the pond water (municipal water with algae) at a constant rate by setting the pressure regulator at a certain pressure. The amount of injected CO₂ was totalized by the digital mass flow meter (Cole-Parmer EW-32908-73). The CO₂ flow rate can be increased or decreased by adjusting the pressure in the line using the pressure regulator downstream of the digital mass flow meter. The built-in PLC recorded the CO₂flow rate on an every minute basis and totalized the time that the CO₂ was on.

The municipal water with algae had a pH range of 8.8 to 9.2, which is lower than the pH of the municipal water.

Below is the protocol that was followed.

1. The optical density of the algae pond culture (municipal water with algae) was brought down to approximately 0.2-0.35, depending on algal productivity. The optical density of the culture can be brought down by harvesting part of the algae in the pond and returning the subnatant from a dissolved air flotation device (DAF) back to the pond, resulting in a decrease in the density of the culture.

2. A total flow of 90 SLPM (standard liter per minute) was fed into a pipe line at a pressure of 30 psi. The CO₂ flow rate was kept constant at 0.87 lb/hr/ft.

3. A sample was taken prior to CO₂ injection and after CO₂ injection. Samples were taken from the pond for total organic carbon (TOC), total carbon (TC), pH, total dissolved solids (TDS), and alkalinity analyses. A hand held meter and an automatic alkalinity measurement instrument were used. Each sample was then analyzed for total organic carbon in a TOC analyzer (InnovOx, GE).

4. The CO₂ was turned on or off by the pH set point of the PLC. For example, for a pH setting range of 8.8-9.2, CO₂ was turned on when the pond was higher than 9.2, and CO₂ was turned off when the pond pH was lower than 8.8. The turning on and off of CO₂ is controlled by a solenoid valve.

5. For 28 days, 1 sample per day was taken just downstream of the paddlewheel, and analyzed 3-4 times.

6. A newly developed CO₂ efficiency calculation equation, described below, was used to calculate the CO₂ efficiency for municipal water with algae in the absence of a sump.

Overall Carbonation Efficiency Calculation

$${CO}_{2} transfer efficiency to biomass ( \% ) = \frac{\begin{matrix}{TOC}_{T} \times ( ( {H}_{T} \times 25.4 - 30 ) \div 0.0017 + 12537.8 ) - \\ {TOC}_{0} \times ( ( {H}_{0} \times 25.4 - 30 ) \div 0.0017 + 12537.8 )\end{matrix}}{{}_{0}^{T}{ΣV}{}_{iCO 2}^{} \times 1.835 \times 1000 \div 44 \div 12} \times 100$$

T: experiment duration (days)

Vᵢ: CO₂ total injection volume at day of injection (Standard liter per minute)

H₀: raceway water depth (inches) at start day (day 0)

H_{T}: raceway water depth (inches) at finish day (day T)

TOC₀: total organic carbon concentration (mg/L) at day 0

TOC_{T}: total organic carbon concentration (mg/L) at finish day (day T)

The above equation was used to calculate the percentage of carbon in CO₂ that is transferred into the biomass. The novel part of this equation is calculating the accumulative biomass increase over accumulative CO₂ injection.

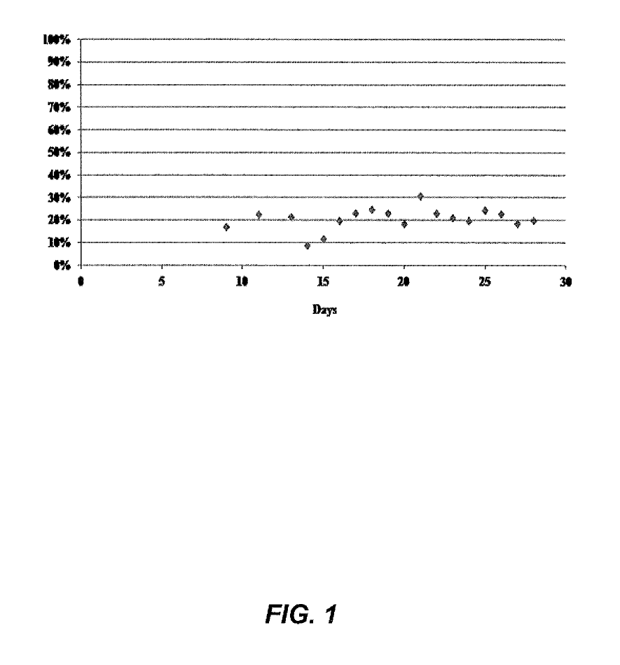

The accumulative overall carbonation efficiency over a period of 28 days is shown in FIG. 1. As shown in FIG. 1, the cumulative overall carbonation efficiency was from about 9% to about 31% over the 28 day period. Pressure loss through the diffuser at the above flow rate was negligible.

### Example 2

### Methods and Experimental Approaches

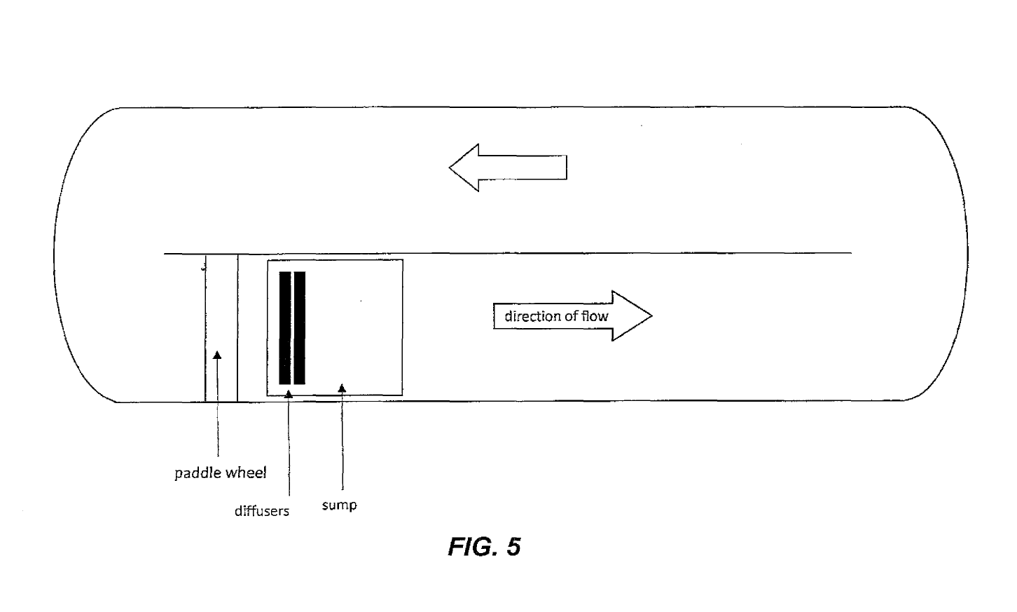

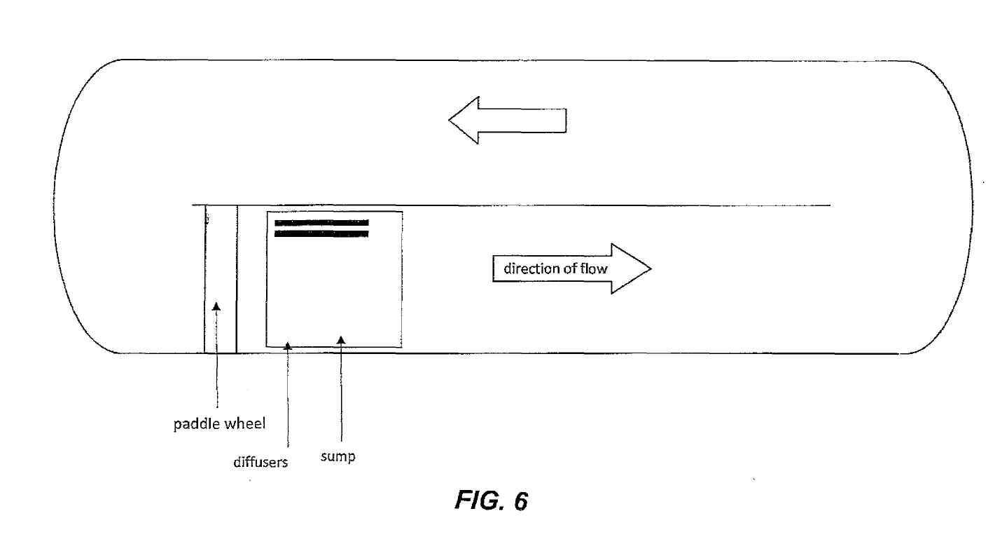

In EXAMPLES 2, 3 and 4, a carbonation sump, as shown in FIG. 2, was built in a 6,400 square foot raceway pond to increase the water depth. Diffusers were set up in three different configurations as shown in FIG. 5 to FIG. 7. The diffusers were located at the bottom of the sump, either along the length of the sump or across the width of the sump. FIG. 5 shows two 9.4 foot long diffusers along the width of the pond. FIG. 6 shows two 9.4 foot long diffusers along the length of the pond. FIG. 7 shows one 25 foot diffuser folder back upon itself located along the width of the pond. Two types of liquids were tested, municipal water and municipal water with algae.

Diffusers

Overall carbonation efficiency testing was conducted using two different diffusers at various CO₂ flow rates. Solvox-B (Solvox, Linde, Germany) diffuser hoses are made of ethylene propylene diene monomer (M-class) rubber (EPDM). The thickness of the wall of the Solvox-B diffuser is 3/16 inch. The inside diameter of the Solvox-B diffuser hose is ⅝ inch. The diffuser hoses were secured at the bottom of the pond using a flat metal frame and connected to a CO₂ feeding line. Aquatech (Aquatech Environmental System Ltd., Canada) diffuser hoses are made of thermal plastic rubber. The thickness of the wall of the Aquatech diffuser is 1/16 inch. The inside diameter of the Aquatech diffuser hose is ½ inch. The diffuser hoses were secured at the bottom of the pond using a U-shaped metal frame and connected to a CO₂ feeding line.

Pressure Drop Measurement

A standard pressure gauge was installed before and after the location of the diffuser hoses to measure the pressure drop along the diffuser hoses at different CO₂ flow rates. For example, in FIG. 7 the two circles at the ends of the U-shaped diffuser hose are pressure gauges.

CO₂ Mass Flow Meter and Programmable Logic Controller (PLC) Set Up

A mobile CO₂ PLC cabinet was located near the 6,400 square ft raceway pond, connecting the diffuser and the CO₂ feed line. The cabinet is composed of a CO₂ digital mass flow meter, a pressure regulator, and a PLC. 100% CO₂ gas was injected into the pond water (either municipal water or municipal water with algae) at a constant rate by setting the pressure regulator at a certain pressure. The amount of injected CO₂ was totalized by the digital mass flow meter (Cole-Parmer EW-32908-73). The CO₂ flow rate can be increased or decreased by adjusting the pressure in the line using the pressure regulator downstream of the digital mass flow meter. The built-in PLC recorded the CO₂ flow rate on an every minute basis and totalized the time that the CO₂ was on.

### Example 3

### Municipal Water

For overall carbonation efficiency testing using municipal water, the pH of the municipal water was brought up to a pH of 10, simulating the desired growing pH of an algal strain. CO₂ was then injected into the water.

Two different diffuser configurations were used to test the municipal water as shown in FIG. 5 and FIG. 6. In addition, both Solvox-B (configured as in FIG. 6) and Aquatech (configured as in FIG. 5) diffusers were tested.

Below is the protocol that was followed.

1. The municipal water was brought to pH 10 with either sodium hydroxide or sodium carbonate and calcium hydroxide.

2. CO₂ was turned on at a fixed flow rate lb/hr/ft diffuser); a range of 0.21 lb/hr/ft to 1.15 lb/hr/ft was tested.

3. The pond was sampled at three different locations for dissolved inorganic carbon (DIC), pH, TDS, and alkalinity. A hand held meter and an automatic alkalinity measurement instrument were used. Samples were taken right after the paddlewheel, at the turning point of the pond, and right before the paddlewheel. Three samples were taken prior to CO₂ injection and three samples were taken after CO₂ injection. Each of the six samples was then analyzed 4-7 times (depending on the relative standard deviation of analysis) for inorganic carbon in a TOC analyzer (InnovOx, GE).

4. The of the water was constantly checked using a handheld meter to determine when to stop the experiment.

5. Injection of CO₂ was stopped when the pH was 1.0 unit lower than the initial pH reading.

6. The CO₂ efficiency calculation equation described below, was used for municipal water to calculate the overall CO₂ efficiency.

Overall Carbonation Efficiency Calculation

The overall carbonation efficiency of municipal water was determined by the following equation:

$$Overall {CO}_{2} efficiency = \frac{( {DIC}_{f} - {DIC}_{0} ) * V}{{C}_{T}} \times 100$$

where DIC_{f}, DiC₀, and C_{T} are defined as the dissolved inorganic carbon concentration in the pond at time finish, dissolved inorganic concentration at time 0 before CO₂ injection, V is the volume of pond water, and C_{T} is the total carbon injected into the pond.

To cross check the DIC measurements taken by the TOC analyzer, a standard method (as described in Standard Methods for the Examination of Water and Wastewater, 21^{st} edition, 2005. ISBN 0-87553-047-8, American Water Works Association, Washington, D.C. 20001-3710) was used to calculate DIC based on alkalinity and pH.

$$[ {HCO}_{3}^{-} ] = \frac{T - 5 \times {10}^{pH - 10}}{1 + 0.94 \times {10}^{pH - 10}} \\ [ {CO}_{3}^{2 -} ] = 0.94 [ {HCO}_{3} ] \times {10}^{pH - 10} \\ [ {CO}_{2} ] = 2 [ {HCO}_{3} ] \times {10}^{6 - pH}$$

$$Total Dissolved {CO}_{2} = [ {CO}_{2} ] + 0.44 ( 2 [ {HCO}_{3} ] + [ {CO}_{3} ] )$$

Where T is the total alkalinity, in mg CaCO₃/L.

In addition, other methods to cross check the DIC measurement by a TOC analyzer were also used. A nomographic and Carbon Dioxide and Forms of Alkalinity by Calculation method were conducted (Standard 4500-B graph). These methods are described in Standard Methods for the Examination of Water and Wastewater, 21^{st} edition, 2005, ISBN 0-87553-047-8, American Water Works Association, Washington, D.C. 20001-3710.

Carbonation Efficiency of Aquatech and Solvox-B Diffusers in Municipal Water

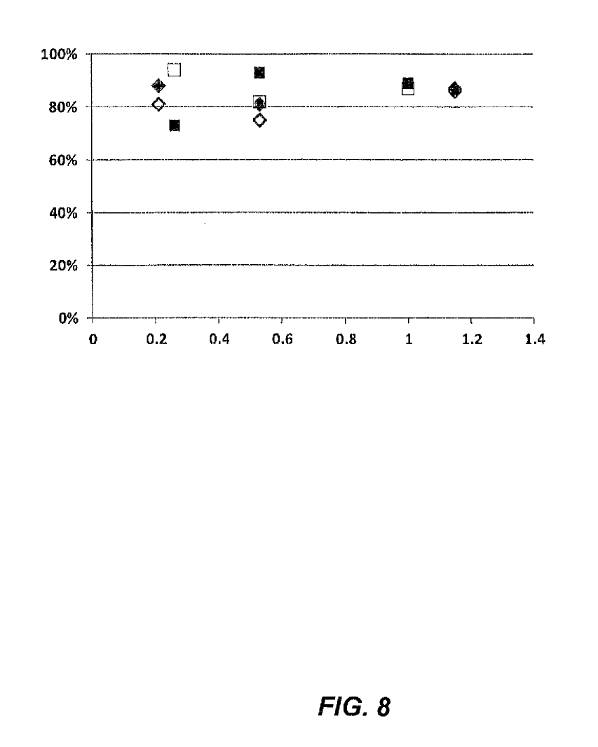

Table 1 and FIG. 8 show the carbonation efficiency of the Aquatech and Solvox-B diffusers at different flow rates at a water depth of 31″ . A range of 81% to 88% efficiency was observed with the Aquatech diffuser with a CO₂ flow rate of 0.21 lb/hr/ft diffuser to 1.15 lb/hr/ft diffuser with direct inorganic carbon measurement. A range of 73% to 93% efficiency was observed with the Solvox-B diffuser with a CO₂ flow rate from 0.26 lb/hr/ft diffuser to 1.00 lb/hr/ft diffuser with direct inorganic carbon measurement. The percent standard deviation is shown in “( )”.

As described above, standard methods for the examination of water and waste water were used to calculate the amount of inorganic carbon based on the alkalinity and the pH of the municipal water and this value was compared to the amount of inorganic carbon measured by the TOC analyzer. The overall carbonation efficiency based on the standard methods is also shown in Table 1 and FIG. 8. For most of the CO₂ flow rate test conditions, the overall carbonation efficiency as determined by the TOC analyzer was consistent with the standard method calculation.

For the Solvox-B, the overall carbonation efficiency increased from 73% to 94% when the CO₂ flow rate increased from 0.26 to 0.53 lb/hr/ft, then the overall carbonation efficiency decreased to 89% when the CO₂ flow rate increased to 1.0 lb/hr/ft. For Aquatech, there was a decrease in the overall carbonation efficiency when the CO₂ flow rate increased from 0.21 to 0.53 lb/hr/ft, and a higher efficiency was observed when the flow rate was further increased to 1.15 lb/hr/ft.

Although better overall carbonation efficiency was observed with the Solvox-B diffuser than with the Aquatech diffuser, some clogging problems were observed with the Solvox-B diffusers, which may cause maintenance issues. Therefore, it was decided to continue the carbonation efficiency tests with the Aquatech diffusers.

A pressure drop of 1-2 psi through the diffuser was observed with both the Aquatech and Solvox-B diffusers at a CO₂ flow rate up to 1.15 lb/hr/ft.

TABLE 1
Overall carbonation efficiency and standard deviations using
Aquatech and Solvox-B diffusers at different CO₂ flow rates.

| | | Aquatech | Solvox-B |
|---|---|---|---|
| | | water | water |
| CO₂ flow | | depth with | depth with |
| rate | | sump is | sump is |
| (lb/hr/ft) | efficiency method | 31 inches | 31 inches |
| 0.21 | % Eff. measure (TOC machine) | 88% (0) | |
| | % Eff. Calc. (Standard 4500 D- | 81% (6%) | |
| | equation) | | |
| | % Eff. Calc. (Standard 4500-B | 76.6% (5%) | |
| | graph) | | |
| 0.26 | % Eff. measure (TOC machine) | | 73% |
| | % Eff. Calc. (Standard 4500 D- | | 94% |
| | equation) | | |
| 0.53 | % Eff. measure (TOC machine) | 81% (4%) | 93% (1%) |
| | % Eff. Calc. (Standard 4500 D- | 75% (3%) | 82% (4%) |
| | equation) | | |
| 1.00 | % Eff. measure (TOC machine) | | 89% |
| | % Eff. Calc. (Standard 4500 D- | | 87% |
| | equation) | | |
| 1.15 | % Eff. measure (TOC machine) | 87% (6%) | |
| | % Eff. Calc. (Standard 4500 D- | NA | |
| | equation) | | |

### Example 4

### Municipal Water with Algae

For overall carbonation efficiency testing using municipal water with algae, a longer period (greater than about two weeks from strain inoculation) was used and algae pond samples were taken on a daily basis.

Below is the protocol that was followed.

1. The optical density of the algae pond culture (municipal water with algae) was brought down to approximately 0.2-0.35, depending on algal productivity. The optical density of the culture can be brought down by harvesting part of the algae in the pond and returning the subnatant from a dissolved air flotation device (DAF) back to the pond, resulting in a decrease in the density of the culture.

2. CO₂ was set at a fixed flow rate; a range of 0.21 lb/hr/ft to 1.15 lb/hr/ft was tested.

3. A sample was taken prior to CO₂ injection and after CO₂ injection. Samples were taken from the pond for TOC, TC, pH, TDs, and alkalinity. A hand held meter and an automatic alkalinity measurement instrument was used. Each sample was then analyzed for total carbon in a TOC analyzer (InnovOx, GE). For the first 8 days, a total of 9 samples were taken at 5 different locations and analyzed 3-4 times. In other words, 9 samples were taken at the same time from 5 locations, at some locations, only one sample was taken, at other locations two samples were taken. After the eighth day, only one sample was taken, from right after the paddlewheel, and analyzed 2-3 times. The five locations were roughly: (1) right after (downstream) of the paddlewheel; (2) in between (1) and right before the first turn in the raceway; (3) the first turn of the raceway; (4) in the middle section of the length of the raceway opposite the side of the paddlewheel; and (5) after the fourth turn but prior to the paddlewheel.

4. The CO₂ was turned on or off by the pH set point of the PLC. For example, for a pH setting range of 8.8-9.2, CO₂ was turned on when the pond pH was higher than 9.2, and CO₂ was turned off when the pond pH was lower than 8.8. The turning on and off of CO₂ is controlled by a solenoid valve.

5. Samples were taken for a period of up to 14 days.

6. A newly developed CO₂ efficiency calculation equation was used to calculate the CO₂ efficiency for municipal water with algae.

Overall Carbonation Efficiency Calculation

The overall carbonation efficiency of municipal water with algae can be determined by the following equation:

$$Overall {CO}_{2} efficiency = \frac{( {TC}_{f} - {TC}_{0} ) * V}{{C}_{T}} \times 100$$

Where TC_{f}, TC₀ and C_{T} are defined as total carbon concentration in the pond at time finish, total carbon concentration at time 0 before CO₂ injection, V is the volume of pond water, and C_{T} is the total carbon injected into the pond.

Instead of the calculations provided directly above, an alternate method to calculate the overall carbonation efficiency of municipal water with algae was conducted using the following equation:

$${CO}_{2} transfer efficiency to biomass ( \% ) = \frac{\begin{matrix}{TOC}_{T} \times ( ( {H}_{T} \times 25.4 - 30 ) \div 0.0017 + 12537.8 ) - \\ {TOC}_{0} \times ( ( {H}_{0} \times 25.4 - 30 ) \div 0.0017 + 12537.8 )\end{matrix}}{{}_{0}^{T}{ΣV}{}_{iCO 2}^{} \times 1.835 \times 1000 \div 44 \div 12} \times 100$$

T: experiment duration (days)

Vᵢ: CO₂ total injection volume at day of injection (Standard liter per minute)

H₀: raceway water depth (inches) at start day (day 0)

H_{T}: raceway water depth (inches) at finish day (day T)

TOC₀: total organic carbon concentration (mg/L) at day 0

TOC_{T}: total organic carbon concentration (mg/L) at finish day (day T)

The above equation was used to calculate the percentage of carbon in CO₂ that is transferred into the biomass. The novel part of this equation is calculating the accumulative biomass increase over accumulative CO₂ injection.

A cultivation pond can have an overall carbonation efficiency of greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, or greater than about 95%. A cultivation pond may have an overall carbonation efficiency of from about 25 to 100%, from about 73 to about 94%, or greater than about 31%.

In addition to calculating the above equation, the same equation was calculated replacing total organic carbon “TOC” with total carbon “TC”. The TOC represents the efficiency of CO₂ into algal biomass, and the TC is the total carbon input into the pond. The TOC should be less than or equal to the TC.

Carbonation Efficiency of Aquatech Diffusers in Municipal Water with Algae

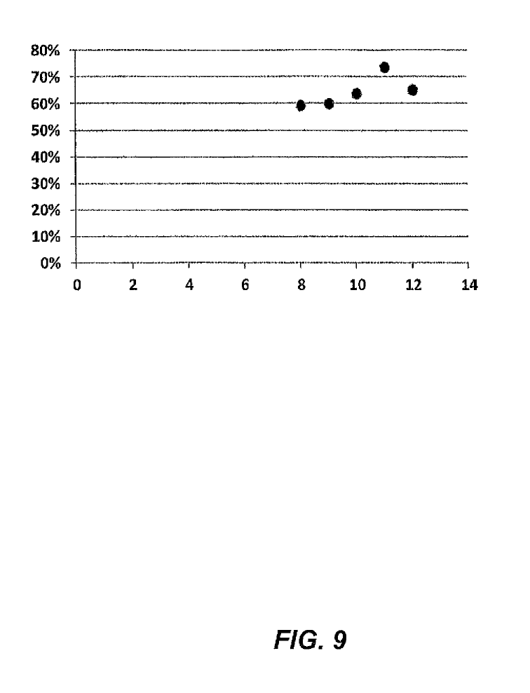

A 25 foot length of Aquatech diffuser was laid out in a 6,400 square foot raceway pond (as shown in FIG. 7). The pressure drop through the 25 foot diffuser was monitored by a pressure gauge (also shown in FIG. 7). A total flow of 90 SLPM (standard liter per minute) was fed into a pipe line at a pressure of 30 psi. The CO₂ flow rate was kept constant at 0.87 lb/hr/ft. The municipal water with algae had a pH range of 8.8 to 9.2, which is lower than the pH of the municipal water. The accumulative overall carbonation efficiency over a period of 14 days is shown in FIG. 9. As shown in FIG. 9, the cumulative overall carbonation efficiency was around 60% at day 8 and increased at day 10 and 11. Pressure loss through the diffuser at the above flow rate was negligible. As shown in FIG. 9, Aquatech diffusers had a 60% overall carbonation efficiency at a CO₂ flow rate of 0.87 lb/hr/ft, 30 inch water depth, and pH 8.8 to 9.2 with a Desmid (Desmodesmus sp.) culture.

### Example 5

### Measurement of Settling or Algal Biomass in a Sump

An algal strain was cultivated in a pond as described in line two of Table B above, with an outflow slope of 20 degrees (L=12 ft, L₁=20 ft, etc.). A 10 foot length of diffuser was laid out in a 610.48 square meter raceway pond (diffuser was laid out as shown in FIG. 4) with a paddlewheel operated at 10 rpm. CO₂ was maintained at a pipe line pressure of 60 psi and regulated by a flow meter at 0.7 g/L prior to being fed to the diffuser. The CO₂ was triggered by an increase in pH above 9.2. The municipal water with algae had a pH range of 8.8 to 9.2.

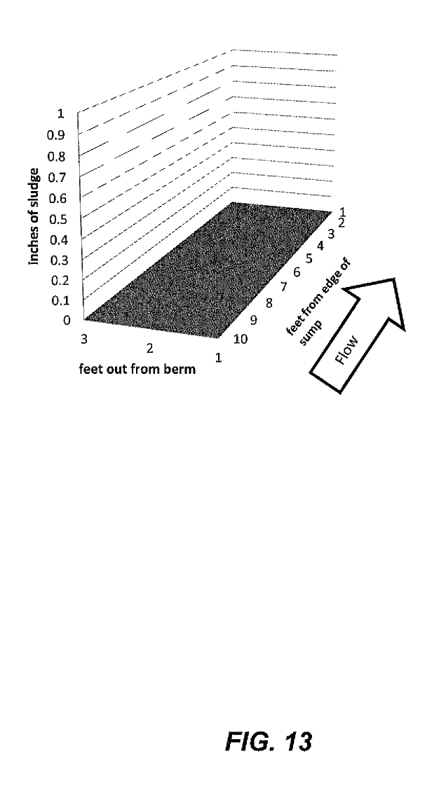

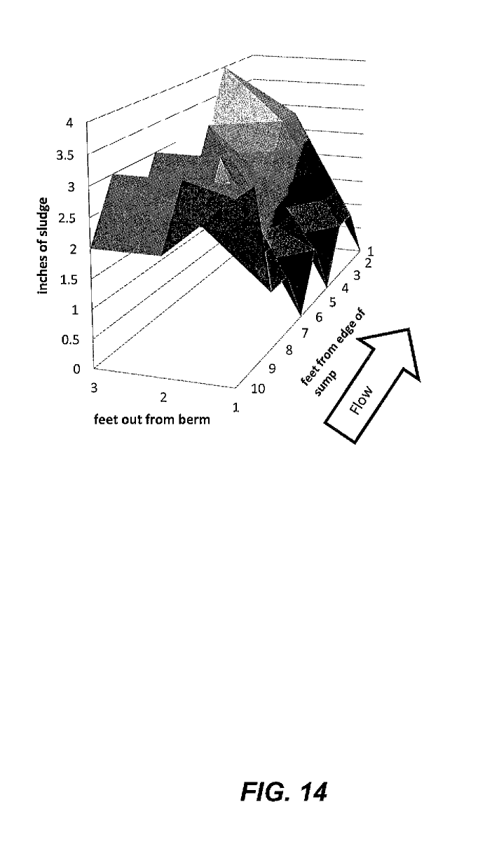

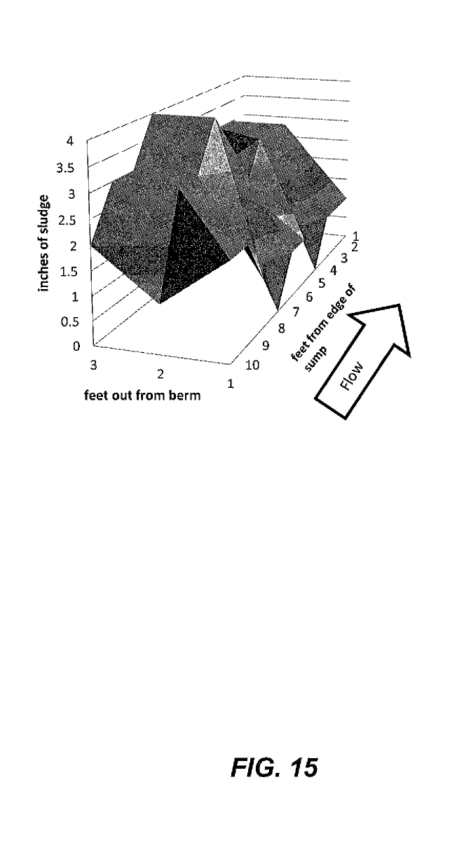

A total of thirty locations within a 10 foot by 3 foot grid (FIG. 12) were sampled with a SludgeProXL Sampler 1¼ inch diameter (“SludgePro”) (Pollardwater, N.Y.) A SludgePro enables accurate readings of settled solids in a variety of liquids at various depths. The grid was started 1 foot from the outside berm (the outer border of the pond) and 1 foot from the beginning of the bottom of the sump. Then SludgePro samples were taken in 1 foot increments up to 3 feet out and in 1 foot increments parallel to the berm. Sludge depths were recorded in inches and compiled to give three-dimensional representations of the sludge (comprising biomass, and any debris, dust, dirt, etc.) accumulations for this area of the sump (FIG. 13, FIG. 14, and FIG. 15). FIG. 13, FIG. 14 and FIG. 15 represent day 5, day 25 and day 26 of a 26 day experiment. The flow of the liquid culture downstream of the paddlewheel is shown by an arrow in all three figures. The average biomass accumulation after 5 days was 0.0, the average biomass accumulation after 25 days was 2.033333, and the average biomass accumulation after 26 days was 2.066667. The data for these averages are presented below in Table 2. Even after 26 days of culturing the strain in a cultivation pond with a sump of the disclosure and average of only 2 inches of sludge was measured. This low level of accumulation of sludge, due to the inflow angle of the sump, greatly minimizes the loss of biomass in the pond resulting in a greater percentage of biomass being recovered.

TABLE 2
Day 5 (FIG. 13) - inches of sludge (comprising biomass)

| | | | | | | | | | | | |
|---|---|---|---|---|---|---|---|---|---|---|---|
| feet | 3 | 0 | 0 | 0 | 0 | 0 | 0 | 0 | 0 | 0 | 0 |
| out | 2 | 0 | 0 | 0 | 0 | 0 | 0 | 0 | 0 | 0 | 0 |
| from | 1 | 0 | 0 | 0 | 0 | 0 | 0 | 0 | 0 | 0 | 0 |
| berm | | | | | | | | | | | |
| | feet | 1 | 2 | 3 | 4 | 5 | 6 | 7 | 8 | 9 | 10 |
| | from | | | | | | | | | | |
| | edge of | | | | | | | | | | |
| | sump | | | | | | | | | | |

Day 25 (FIG. 14) - inches of sludge (comprising biomass)

| | | | | | | | | | | | |
|---|---|---|---|---|---|---|---|---|---|---|---|
| feet | 3 | 2 | 3 | 4 | 3 | 2 | 2 | 3 | 2 | 3 | 2 |
| out | 2 | 2 | 3 | 3 | 2 | 2 | 2 | 3 | 2 | 3 | 2 |
| from | 1 | 0 | 1 | 1 | 0 | 1 | 0 | 1 | 1 | 3 | 3 |
| berm | | | | | | | | | | | |
| | feet | 1 | 2 | 3 | 4 | 5 | 6 | 7 | 8 | 9 | 10 |
| | from | | | | | | | | | | |
| | edge of | | | | | | | | | | |
| | sump | | | | | | | | | | |

Day 26 (FIG. 15) - inches of sludge (comprising biomass)

| | | | | | | | | | | | |
|---|---|---|---|---|---|---|---|---|---|---|---|
| feet | 3 | 2 | 3 | 3 | 2 | 2 | 2 | 4 | 3 | 3 | 2 |
| out | 2 | 2 | 3 | 3 | 3 | 2 | 2 | 4 | 3 | 3 | 1 |
| from | 1 | 1 | 1 | 1 | 0 | 1 | 1 | 0 | 1 | 2 | 2 |
| berm | | | | | | | | | | | |
| | feet | 1 | 2 | 3 | 4 | 5 | 6 | 7 | 8 | 9 | 10 |
| | from | | | | | | | | | | |
| | edge of | | | | | | | | | | |
| | sump | | | | | | | | | | |

It should be noted that for any and all equations described herein one skilled in the art could easily change/convert any of the units described, for example, change liters per minute to liters per hours, change mg/L to g/L, change water depth from inches to centimeters, or change day to hours.

It should also be noted that the overall carbonation efficiency of other organisms, like cyanobacteria can also be determined using the embodiments described herein. One skilled in the art would know how to adjust the equations provided above, if needed, to obtain accurate data.

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A pond for the cultivation of a non-vascular photosynthetic organism in a liquid medium, comprising:
a) a sump comprising an inflow slope with an angle of from about 20 degrees to about 70 degrees:
b) an outflow slope with an angle of from about 5 degrees to about 40 degrees;
c) a back flow region of the inflow slope having a volume that is less than about 50% of the total volume of the sump;
d) a depreciation depth of from about 10 inches to about 3 inches; and
e) a carbon dioxide input that injects carbon dioxide into said liquid medium, wherein the pond has an overall carbonation efficiency of greater than about 20%.

2-5. (canceled)

6. The pond of claim 1, wherein the inflow slope has an angle of about 30 degrees and the outflow slope has an angle of about 20 degrees.

7. The pond of claim 1, wherein the inflow slope has an angle of from about 20 degrees to about 60 degrees, from about 20 degrees to about 40 degrees, about 20 degrees, about 25 degrees, about 30 degrees, about 35 degrees, about 40 degrees, about 45 degrees, about 50 degrees, about 55 degrees, about 60 degrees, about 65 degrees, or about 70 degrees.

8. The pond of claim 1, wherein the outflow slope has an angle of from about 20 degrees to about 40 degrees.

9. (canceled)

10. The pond of claim 1, wherein the pond has an overall carbonation efficiency of from about 73% to about 94%, from about 40% to about 90%, from about 81% to about 88%, from about 73% to about 93%, from about 30% to about 70%, about 60%, or about 89%.

11. (canceled)

12. (canceled)

13. The pond of claim 1, wherein the depreciation depth is about 21 inches or about 25 inches.

14. The pond of claim 1, wherein the sump has a water depth of from about 8 inches to about 40 inches.

15. The pond of claim 1, wherein the water depth is about 21 inches, about 30 inches, or about 31 inches.

16. The pond of claim 1, further comprising a fluid distribution system located upstream of the inflow slope.

17. The pond of claim 16, wherein the fluid distribution system is a paddle wheel.

18. (canceled)

19. (canceled)

20. The pond of claim 16, wherein the fluid distribution system is a jet circulation system.

21-51. (canceled)

52. The pond of claim 1, wherein the carbon dioxide input results in a carbon dioxide flow rate of from about 0.0022 lb/hr/feet to about 1.15 lb/hr/feet or from about 0.0022 to about 2.0 lb/hr/feet.

53. The pond of claim 1, wherein the carbon dioxide input results in a carbon dioxide flow rate of from about 0.21 lb/hr/feet to about 1.15 lb/hr/feet.

54. The pond of claim 53, wherein the carbon dioxide input results in a carbon dioxide flow rate of from about 0.26 lb/hr/feet to about 1.0 lb/hr/feet.

55. (canceled)

56. (canceled)

57. The pond of claim 1, wherein the carbon dioxide input is a diffuser.

58. The pond of claim 57, wherein the diffuser is at least one diffuser hose.

59. The pond of claim 58, wherein the at least one diffuser hose is located along the length (L) of the sump.

60. (canceled)

61. (canceled)

62. The pond of claim 1, wherein the carbon dioxide input is a nozzle.

63-65. (canceled)

66. A method of growing a non-vascular photosynthetic organism, comprising:
a) obtaining the non-vascular photosynthetic organism;
b) obtaining a pond as described in claim 1; and
c) growing the non-vascular photosynthetic organism in the pond.

67. The method of claim 66, wherein the non-vascular photosynthetic organism is an alga or a cyanobacterium.

68-81. (canceled)
